(19) United States
(12) Patent Application Publication
(10) Pub. No.: US 2025/0057793 A1
(43) Pub. Date: Feb. 20, 2025

(54) BENZOIC ACID SALTS FOR TREATMENT OF NERVOUS SYSTEM INJURIES AND DISORDERS

(71) Applicants: The United States Government as represented by the Department of Veterans Affairs, Washington, DC (US); Rush University Medical Center, Chicago, IL (US)

(72) Inventor: Kalipada Pahan, Chicago, IL (US)

(21) Appl. No.: 18/720,456

(22) PCT Filed: Dec. 15, 2022
(86) PCT No.: PCT/US2022/053034

§ 371 Date: Jun. 14, 2024

Related U.S. Application Data
(60) Provisional application No. 63/290,633, filed on Dec. 16, 2021.

(51) Int. Cl. A61K 31/192; A61K 31/045; A61K 31/198; A61K 31/216; A61K 31/222; A61P 25/00; A61P 25/28

(52) U.S. Cl. CPC A61K 31/192; A61K 31/045; A61K 31/198; A61K 31/216; A61K 31/222; A61P 25/00; A61P 25/28

(57) ABSTRACT

Disclosed are salts of benzoic acid and prodrugs thereof for slowing the progression of or reducing the severity of a symptom associated with a nervous system injury in a subject.

### CROSS-REFERENCE TO RELATED APPLICATION

This application claims priority to U.S. Provisional Application No. 63/290,633 filed Dec. 16, 2021, which is incorporated by reference in its entirety.

### BACKGROUND

Annually, about 1.7 million of people in the U.S. and 10 million people globally suffer from traumatic brain injury (TBI). TBI is a major cause of death and disability in the U.S. and contributes to about 30% of all injury-related deaths. Although all people are at risk, military personnel are at greater risk of TBI due to the nature of their profession. During TBI, chronic neuroinflammation and demyelination and/or remyelination failure are important contributors of disability among military personnel. Although there are treatments to reduce or eliminate certain physical, emotional, and cognitive problems associated with TBI, an effective neuroprotective therapy is needed for a person to recover from TBI.

Following TBI and other nervous system injuries, a series of complex pathophysiological events occurs, causing both structural damage and functional deficits. Activation of glial cells and associated upregulation of proinflammatory molecules in the nervous system participate in the pathogenesis of a number of neurodegenerative and neuroinflammatory diseases. Accordingly, one of the main hallmarks of both acute and chronic TBI is also neuroinflammation, which is evidenced within minutes of TBI. Studies from laboratory animals of focal and diffuse TBI have shown the involvement of various proinflammatory molecules such as IL-10, TNF-α and inducible nitric oxide synthase (iNOS) in the pathogenesis of TBI. Many clinical studies demonstrated the increases in IL-10 and TNF-α in CSF and serum of TBI patients as compared to healthy controls. Upregulation of broad-spectrum proinflammatory molecules in the brain causes edema, blood brain barrier (BBB) leakage, neuronal apoptosis, and atrophy, eventually leading to functional impairments. A need exists for more effective treatments for injuries to the nervous system, including TBI.

### SUMMARY

In one aspect, the method of slowing the progression of or reducing the severity of a symptom associated with a nervous system injury in a subject in need thereof comprises administering to the subject an effective amount of a benzoic acid salt or a prodrug thereof, thereby slowing the progression of or reducing the severity of the symptom associated with the nervous system injury.

In another aspect, a method of slowing the progression of or reducing the severity of a symptom associated with a nervous system injury in a subject in need thereof comprises administering to the subject an effective amount of sodium benzoate, thereby slowing the progression of or reducing the severity of the symptom associated with the nervous system injury.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1A is a photograph of the CCI machine tip with mouse brain exposed. Using the CCI technique, brain injury was gently induced onto the exposed brain region of anesthetized mice.

FIG. 1B is a photograph showing CCI induced in the brain. Blood clots and tissue damage in burr hole (stereotactic coordinates—from bregma 1.5 mm posterior and 1.5 mm lateral) were seen in the injured brain region of mice after CCI injury.

FIG. 1C are images showing the induction of mild, moderate, and severe CCI injury using a 1 mm tip with three different velocities, viz. 1.0V, 1.25V and 1.5V respectively. After one-week post-injury, mice (n=3) were perfused with 4% paraformaldehyde followed by removal of brains and staining the brain sections with cresyl violet.

FIG. 1D is a schematic of the experimental design showing the time course of treatment, behavioral, and histological analysis following CCI injury (1 mm tip/1.0V).

FIG. 2A are images of double-label immunofluorescence for GFAP and iNOS in brain sections for the control. Mice were treated with 50 mg/kg/day of NaB or NaFO via oral administration after the induction of CCI injury. After 7 days of NaB treatment, brain sections were analyzed by double-label immunofluorescence for GFAP and iNOS. These results show that oral treatment of NaB attenuates the activation of astrocytes in vivo in the cortex and hippocampus region of mice with CCI injury.

FIG. 2B are images of double-label immunofluorescence for GFAP and iNOS in brain sections for CCI injury. Mice were treated with 50 mg/kg/day of NaB or NaFO via oral administration after the induction of CCI injury. After 7 days of NaB treatment, brain sections were analyzed by double-label immunofluorescence for GFAP and iNOS. These results show that oral treatment of NaB attenuates the activation of astrocytes in vivo in the cortex and hippocampus region of mice with CCI injury.

FIG. 2C are images of double-label immunofluorescence for GFAP and iNOS in brain sections for CCI+NaB. Mice were treated with 50 mg/kg/day of NaB or NaFO via oral administration after the induction of CCI injury. After 7 days of NaB treatment, brain sections were analyzed by double-label immunofluorescence for GFAP and iNOS. These results show that oral treatment of NaB attenuates the activation of astrocytes in vivo in the cortex and hippocampus region of mice with CCI injury.

FIG. 2D are images of double-label immunofluorescence for GFAP and iNOS in brain sections for CCI+NaFO. Mice were treated with 50 mg/kg/day of NaB or NaFO via oral administration after the induction of CCI injury. After 7 days of NaB treatment, brain sections were analyzed by double-label immunofluorescence for GFAP and iNOS. These results show that oral treatment of NaB attenuates the activation of astrocytes in vivo in the cortex and hippocampus region of mice with CCI injury.

FIG. 2E is a histogram of cells positive for GFAP counted in the cortex region. Results represent analysis of six sections of each of six mice per group. ^{a}p<0.001 vs control; ^{b}p<0.001 vs CCI injury.

FIG. 2F is a histogram of cells positive for GFAP counted in the CA1 region. Results represent analysis of six sections of each of six mice per group. ^{a}p<0.001 vs control; ^{b}p<0.001 vs CCI injury.

FIG. 2G is a histogram of cells positive for iNOS counted in the cortex region. Results represent analysis of six sections of each of six mice per group. ^{a}p<0.001 vs control; ^{b}p<0.001 vs CCI injury.

FIG. 2H is a histogram of cells positive for iNOS counted in the CA1 region. Results represent analysis of six sections of each of six mice per group. ^{a}p<0.001 vs control; ^{b}p<0.001 vs CCI injury.

FIG. 2I is an immunoblot image of tissue extracts of hippocampal region from all groups of mice (n=4 per group) for GFAP. Actin was run as a loading control.

FIG. 2J is a plot showing the values of GFAP/Actin relative to control as obtained by immunoblot band scanning. ^{a}p<0.001 vs control; ^{b}p<0.001 vs CCI injury.

FIG. 3A are images of double-label fluorescence for Iba1 and iNOS for the control. Mice were treated with 50 mg/kg/day of NaB or NaFO from 24 hrs after the induction of CCI injury. After 7 days of treatment, brain sections were analyzed by double-label fluorescence for Iba1 and iNOS. These results show that NaB treatment inhibits microglial activation in vivo in the cortex and hippocampus of mice with CCI injury.

FIG. 3B are images of double-label fluorescence for Iba1 and iNOS for CCI injury. Mice were treated with 50 mg/kg/day of NaB or NaFO from 24 hrs after the induction of CCI injury. After 7 days of treatment, brain sections were analyzed by double-label fluorescence for Iba1 and iNOS. These results show that NaB treatment inhibits microglial activation in vivo in the cortex and hippocampus of mice with CCI injury.

FIG. 3C are images of double-label fluorescence for Iba1 and iNOS for CCI+NaB. Mice were treated with 50 mg/kg/day of NaB or NaFO from 24 hrs after the induction of CCI injury. After 7 days of treatment, brain sections were analyzed by double-label fluorescence for Iba1 and iNOS. These results show that NaB treatment inhibits microglial activation in vivo in the cortex and hippocampus of mice with CCI injury.

FIG. 3D are images of double-label fluorescence for Iba1 and iNOS for CCI+NaFO. Mice were treated with 50 mg/kg/day of NaB or NaFO from 24 hrs after the induction of CCI injury. After 7 days of treatment, brain sections were analyzed by double-label fluorescence for Iba1 and iNOS. These results show that NaB treatment inhibits microglial activation in vivo in the cortex and hippocampus of mice with CCI injury.

FIG. 3E is a histogram of cells positive for Iba1 counted in the cortex region. Results represent analysis of six sections of each of six mice per group. ^{a}p<0.001 vs control; ^{b}p<0.001 vs CCI injury.

FIG. 3F is a histogram of cells positive for Iba1 counted in the CA1 region. Results represent analysis of six sections of each of six mice per group. ^{a}p<0.001 vs control; ^{b}p<0.001 vs CCI injury.

FIG. 3G is an immunoblot image of tissue extracts of hippocampal region from all groups of mice (n=4 per group) for Iba1. Actin was run as a loading control.

FIG. 3H is a plot showing the values of Iba1/Actin relative to control as obtained by immunoblot band scanning. ^{a}p<0.001 vs control; ^{b}p<0.001 vs CCI injury.

FIG. 3I is an immunoblot image of tissue extracts of hippocampal region from all groups of mice (n=4 per group) for iNOS. Actin was run as a loading control.

FIG. 3J is a plot showing the values of iNOS/Actin relative to control as obtained by immunoblot band scanning. ^{a}p<0.001 vs control; ^{b}p<0.001 vs CCI injury.

FIG. 4 are images of the corpus collosum in mice showing the levels of proteolipid protein (PLP) and A2B5, a marker of oligodendroglial progenitor cells (OPC), in the experimental conditions after 21 days. Oral administration of sodium benzoate (NaB), but not sodium formate (NaFO), stimulates remyelination in mice with traumatic brain injury (TBI). Mice (n=6 per group) were induced moderate TBI by controlled cortical impact (CCI). Starting from 2 hours after TBI, mice were treated with NaB and NaFO (50 mg/kg body weight/day; mixed with water) orally via gavage. After 21 d of treatment, brain sections were double-labeled for PLP and A2B5. Results represent analysis of two sections of each of six mice per group.

FIG. 5A are images showing representative cresyl violet sections of mouse brain arranged in series of hippocampal region shows the volume of lesion cavity in different groups. NaB treatment reduces the lesion volume in mice with CCI injury.

FIG. 5B are illustrative images of cresyl violet section. Note the extent of damage induced in brain was found to be reduced in NaB treated mice when compared to CCI-mice without treatment and NaFo treated CCI injury mice.

FIG. 5C is a plot showing lesion size in the experimental conditions. Lesion size was quantitatively measured in control mice, untreated CCI injured mice, NaB treated CCI-mice and NaFO-treated CCI-mice at 21 days post-injury. Statistical analyses were performed with Student's t-test in the injured side of the brain [^{a}p<0.001 (5.623×10⁻⁷) vs control; ^{b}p<0.001 (=0.001) vs CCI injury].

FIG. 6A shows a heat map analysis for mice (n=6 per group) with TBI induced by CCI in open field activities after 7 days of treatment. Open field activities were monitored by the Ethovision XT 13.0 Open Field Activity System (Noldus). Starting from 2 hours after TBI, mice were treated with NaB and NaFO (50 mg/kg body weight/day; mixed with water) orally via gavage. Oral administration of sodium benzoate (NaB) but not sodium formate (NaFO) improves open field activities in mice with TBI.

FIG. 6B is a bar graph showing the distance traveled by mice (n=6 per group) with TBI induced by CCI in open field activities after 7 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10⁻⁶) vs control; ^{b}p<0.001 (=0.0081) vs CCI injury].

FIG. 6C is a bar graph showing the velocity of mice (n=6 per group) with TBI induced by CCI in open field activities after 7 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10⁻⁶) vs control; ^{b}p<0.001 (=0.0081) vs CCI injury].

FIG. 6D is a bar graph showing the center frequency of mice (n=6 per group) with TBI induced by CCI in open field activities after 7 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10⁻⁶) vs control; ^{b}p<0.001 (=0.0081) vs CCI injury].

FIG. 6E is a bar graph showing the rearing behavior of mice (n=6 per group) with TBI induced by CCI in open field activities after 7 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10−) vs control; ^{b}p<0.001 (=0.0081) vs CCI injury].

FIG. 6F shows heat map analysis for mice (n=6 per group) with TBI induced by CCI in open field activities after 21 days of treatment. Open field activities were monitored by the Ethovision XT 13.0 Open Field Activity System (Noldus). Starting from 2 hours after TBI, mice were treated with NaB and NaFO (50 mg/kg body weight/day; mixed with water) orally via gavage.

FIG. 6G is a bar graph showing the distance traveled by mice (n=6 per group) with TBI induced by CCI in open field activities after 21 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10⁻⁶) vs control; ^{b}p<0.001 (=0.0081) vs CCI injury].

FIG. 6H is a bar graph that shows the velocity of mice (n=6 per group) with TBI induced by CCI in open field activities after 21 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10−) vs control; ^{b}p<0.001 (=0.0081) vs CCI injury].

FIG. 6I is a bar graph showing the center frequency of mice (n=6 per group) with TBI induced by CCI in open field activities after 21 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10⁻⁶) vs control; ^{b}p<0.001 (=0.0081) vs CCI injury].

FIG. 6J is a bar graph showing the rearing of mice (n=6 per group) with TBI induced by CCI in open field activities after 21 days of treatment. Statistical analyses were conducted with Student t-test for distance moved [^{a}p<0.001 (=0.0001) vs control; ^{b}p<0.001 (=0.0029) vs CCI injury]; velocity [^{a}p<0.001 (=0.0001) vs control; ^{b}p<0.001 (=0.0078) vs CCI injury]; center frequency [^{a}p<0.001 (=0.0001) vs control; ^{b}p<0.001 (=0.0036) vs CCI injury] and rearing behavior [^{a}p<0.001 (=9.498×10⁻⁶) vs control; ^{b}p<0.001 (=0.0081) vs CCI injury].

FIG. 6K is a bar graph showing the results for tail suspension test. Following the NaB treatment, mice with CCI-injury showed significant improvement in tail suspension test on 7-day post-injury [^{a}p<0.001 (=5.725×10⁻⁸) vs control; ^{b}p<0.001 (=0.0001) vs CCI injury] and 21-day post-injury [^{a}p<0.001 (=3.995×10⁻⁵) vs control; ^{b}p<0.001 (=0.0003) vs CCI injury].

FIG. 6L is a bar graph showing the results for rotarod test. The performance of mice with NaB treatment has significantly improved in the rotarod test on 7-day post-injury [^{a}p<0.001 (=9.5998×10⁻⁹) vs control; ^{b}p<0.001 (=0.0003) vs CCI injury] and 21-day post-injury [^{a}p<0.001 (=1.2133×10⁻⁹) vs control; ^{b}p<0.001 (=0.0010) vs CCI injury].

FIG. 6M is a bar graph showing the number of steps on beam runway. Oral treatment of NaB also improved the performance of CCI-injured mice on beam runway. Statistical significance was performed using the Student t-test on 7-day post-injury [Steps: ^{a}p<0.001 (=2.258×10⁻⁶) vs control; ^{b}p<0.001 (=0.0027) vs CCI injury; Time taken: ^{a}p<0.001 (=2.979×10⁻⁷) vs control; ^{b}p<0.001 (=0.0039) vs CCI injury; Foot slipping: ^{a}p<0.001 (=1.567×10⁻⁷) vs control; ^{b}p<0.001 (=0.0001) vs CCI injury] and on 21-day post-injury [Steps: ^{a}p<0.001 (=0.0051) vs control; ns (=0.2533) vs CCI injury; Time taken: ^{a}p<0.001 (=0.0003) vs control; ns (=0.1228) vs CCI injury; Foot slipping: ^{a}p<0.001 (=0.0001) vs control; ^{b}p<0.05 (=0.0736) vs CCI injury].

FIG. 6N is a bar graph showing the time taken on beam runway. Oral treatment of NaB also improved the performance of CCI-injured mice on beam runway. Statistical significance was performed using the Student t-test on 7-day post-injury [Steps: ^{a}p<0.001 (=2.258×10⁻⁶) vs control; ^{b}p<0.001 (=0.0027) vs CCI injury; Time taken: ^{a}p<0.001 (=2.979×10⁻⁷) vs control; ^{b}p<0.001 (=0.0039) vs CCI injury; Foot slipping: ^{a}p<0.001 (=1.567×10⁻⁷) vs control; ^{b}p<0.001 (=0.0001) vs CCI injury] and on 21-day post-injury [Steps: ^{a}p<0.001 (=0.0051) vs control; ns (=0.2533) vs CCI injury; Time taken: ^{a}p<0.001 (=0.0003) vs control; ns (=0.1228) vs CCI injury; Foot slipping: ^{a}p<0.001 (=0.0001) vs control; ^{b}p<0.05 (=0.0736) vs CCI injury].

FIG. 6O is a bar graph showing the foot-slipping on beam runway. Oral treatment of NaB also improved the performance of CCI-injured mice on beam runway. Statistical significance was performed using the Student t-test on 7-day post-injury [Steps: ^{a}p<0.001 (=2.258×10⁻⁶) vs control; ^{b}p<0.001 (=0.0027) vs CCI injury; Time taken: ^{a}p<0.001 (=2.979×10⁻⁷) vs control; ^{b}p<0.001 (=0.0039) vs CCI injury; Foot slipping: ^{a}p<0.001 (=1.567×10⁻⁷) vs control; ^{b}p<0.001 (=0.0001) vs CCI injury] and on 21-day post-injury [Steps: ^{a}p<0.001 (=0.0051) vs control; ns (=0.2533) vs CCI injury; Time taken: ^{a}p<0.001 (=0.0003) vs control; ns (=0.1228) vs CCI injury; Foot slipping: ^{a}p<0.001 (=0.0001) vs control; ^{b}p<0.05 (=0.0736) vs CCI injury].

FIG. 6P is a bar graph showing number of steps in grid runway. CCI injured mice with NaB treatment exhibited improvements in grid runway. Using Student t-test, statistical significance were performed on 7-day post-injury [Steps: ^{a}p<0.001 (=9.364×10⁻⁹) vs control; ^{b}p<0.001 (3.394×10⁻⁵) vs CCI injury; Time taken: ^{a}p<0.001 (=1.3770×10˜⁷) vs control; ^{b}p <0.001 (=3.3886×10⁻⁵) vs CCI; Foot-misplacement: ^{a}p<0.001 (=2.737×10⁻⁸) vs control; ^{b}p<0.001 (=5.954×10⁻⁶) vs CCI injury] and on 21-day post-injury (Steps: ^{a}p<0.001 (=0.0014) vs control; ^{b}p<0.05 (=0.0718) vs CCI injury; Time taken: ^{a}p<0.001 (=5.562×10⁻⁵) vs control; ^{b}p<0.05 (=0.072) vs CCI injury; Foot-misplacement: ^{a}p<0.001 (=1.079×10⁻⁵) vs control; ns (=0.2465) vs CCI injury. ns—Non-significant.

FIG. 6Q is a bar graph showing time taken in grid runway. CCI injured mice with NaB treatment exhibited improvements in grid runway. Using Student t-test, statistical significance were performed on 7-day post-injury [Steps: ^{a}p<0.001 (=9.364×10⁻⁹) vs control; ^{b}p<0.001 (=3.394×10⁻⁵) vs CCI injury; Time taken: ^{a}p<0.001 (=1.3770×10⁻⁷) vs control; ^{b}p<0.001 (=3.3886×10⁻⁵) vs CCI; Foot-misplacement: ^{a}p<0.001 (=2.737×10⁻⁸) vs control; ^{b}p<0.001 (=5.954×10⁻⁶) vs CCI injury] and on 21-day post-injury (Steps: ^{a}p<0.001 (=0.0014) vs control; ^{b}p<0.05 (=0.0718) vs CCI injury; Time taken: ^{a}p<0.001 (=5.562×10⁻⁵) vs control; ^{b}p<0.05 (=0.072) vs CCI injury; Foot-misplacement: ^{a}p<0.001 (=1.079×10⁻⁵) vs control; ns (=0.2465) vs CCI injury. ns—Non-significant.

FIG. 6R is a bar graph showing foot misplacement in grid runway. CCI injured mice with NaB treatment exhibited improvements in grid runway. Using Student t-test, statistical significance were performed on 7-day post-injury [Steps: ^{a}p<0.001 (=9.364×10⁻⁹) vs control; ^{b}p<0.001 (=3.394×10⁻⁵) vs CCI injury; Time taken: ^{a}p<0.001 (=1.3770×10⁻⁷) vs control; ^{b}p<0.001 (=3.3886×10⁻⁵) vs CCI; Foot-misplacement: ^{a}p<0.001 (=2.737×10⁻⁸) vs control; ^{b}p<0.001 (=5.954×10⁻⁶) vs CCI injury] and on 21-day post-injury (Steps: ^{a}p<0.001 (=0.0014) vs control; ^{b}p<0.05 (=0.0718) vs CCI injury; Time taken: ^{a}p<0.001 (=5.562×10⁻⁵) vs control; ^{b}p<0.05 (=0.072) vs CCI injury; Foot-misplacement: ^{a}p<0.001 (=1.079×10⁻⁵) vs control; ns (=0.2465) vs CCI injury. ns—Non-significant.

FIG. 7A shows heat maps demonstrating the novel object recognition of mice (n=6 per group) with TBI induced by CCI 21 days post-operation. Starting from 2 hours after TBI, mice were treated with NaB and NaFO (50 mg/kg body weight/day; mixed with water) orally via gavage. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIG. 7B shows heat maps demonstrating the Barnes circular maze test results of mice (n=6 per group) with TBI induced by CCI 21 days post-operation. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIG. 7C is a bar graph showing the exploration time of mice (n=6 per group) with TBI induced by CCI 21 days post-operation during novel object recognition test. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIG. 7D is a bar graph showing the latency time of mice (n=6 per group) with TBI induced by CCI 21 days post-operation during the Barnes maze test. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIG. 7E is a bar graph showing the number of errors of mice (n=6 per group) with TBI induced by CCI 21 days post-operation during the Barnes maze test. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIG. 7F is a bar graph showing the number of positive turns of mice (n=6 per group) with TBI induced by CCI 21 days post-operation during the T maze. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIG. 7G is a bar graph showing the number of negative turns of mice (n=6 per group) with TBI induced by CCI 21 days post-operation during the T maze. Statistical analyses were performed by Student t-test for Novel object recognition test [Exploration time: ^{a}p<0.001 (=2.5989×10⁻⁵) vs control; ^{b}p<0.001 (=0.0003) vs CCI injury]; Barnes maze test [Time taken: ^{a}p<0.001 (=1.7509×10⁻⁵) vs control; ^{b}p<0.001 (=4.8824×10⁻⁵) vs CCI injury and Number of errors: ^{a}p<0.001 (=3.234×10⁻⁵) vs control; ^{b}p<0.001 (=0.0001) vs CCI injury]; T-maze [Positive turns: ^{a}p<0.001 (=3.3524×10⁻⁵) vs control; ^{b}p<0.001 (=0.0004) vs CCI injury and Negative turns: ^{a}p<0.001 (=3.3924×10⁻⁵) vs control; ^{b}p<0.001 (=0.0005) vs CCI injury]. ns—Non-significant.

FIGS. 8A-8D are images of double-label immunofluorescence for GFAP and iNOS in brain sections for the control (FIG. 8A), CCI (FIG. 8B), CCI+GTB (FIG. 8C) and CCI+Vehicle (FIG. 8D. Mice were treated with 50 mg/kg/day of GTB via oral gavage after the induction of CCI injury. After 7 days of GTB treatment, brain sections were analyzed by double-label immunofluorescence for GFAP and iNOS. Cells positive for GFAP were counted in cortex (FIG. 8E) and CA1 region of hippocampus (FIG. 8F). Similarly, cells positive for iNOS were also counted in cortex (FIG. 8G) and CA1 region (FIG. 8H). Results represent analysis of six sections of each of six mice per group. Tissue extracts of hippocampal region from all groups of mice (n=4 per group) were immunoblotted for GFAP (FIG. 8I) and iNOS (FIG. 8K). Actin was run as a loading control. Bands were scanned, and values (GFAP/Actin) (FIG. 8J) and (iNOS/Actin) (FIG. 8L) presented as relative to control. These results show that oral administration of GTB inhibits astroglial inflammation in vivo in the cortex and hippocampus of mice with TBI.

FIGS. 9A-9H. Oral GTB decreases microglial activation in vivo in the cortex and hippocampus of mice with TBI. TBI was induced in mice by CCI injury and after 24 h of injury, mice were treated with 50 mg/kg/day of GTB via oral gavage. Seven days after GTB treatment, brain sections were double-labeled for Iba1 and iNOS (FIG. 9A, control; FIG. 9B, CCI; FIG. 9C, CCI+GTB; FIG. 9D, CCI+Vehicle). Cells positive for Iba1 were counted in cortex (FIG. 9E) and CA1 region of hippocampus (FIG. 9F). Results represent analysis of two sections of each of six mice per group. Tissue extracts of hippocampal region from all groups of mice (n=4 per group) were immunoblotted for Iba1 (FIG. 9G). Actin was run as a loading control. Bands were scanned, and values (Iba1/Actin) (FIG. 9H) presented as relative to control.

FIGS. 10A-10C. Decrease in lesion volume in TBI mice by GTB treatment. TBI was induced in mice by CCI injury and after 24 h of injury, mice were treated with 50 mg/kg/day of GTB via oral gavage. FIG. 10A. Twenty-one days after injury, brain sections were stained with H&E and H&E stained sections were arranged in a series demonstrating the volume of lesion cavity in different groups. FIG. 10B shows illustrative images of H&E stained sections. FIG. 10C. Lesion volume was quantified in all groups of mice. Statistical analyses were performed with two way ANOVA and expressed as mean±SD to compare the lesion volume between unlesioned and lesioned side of the brain.

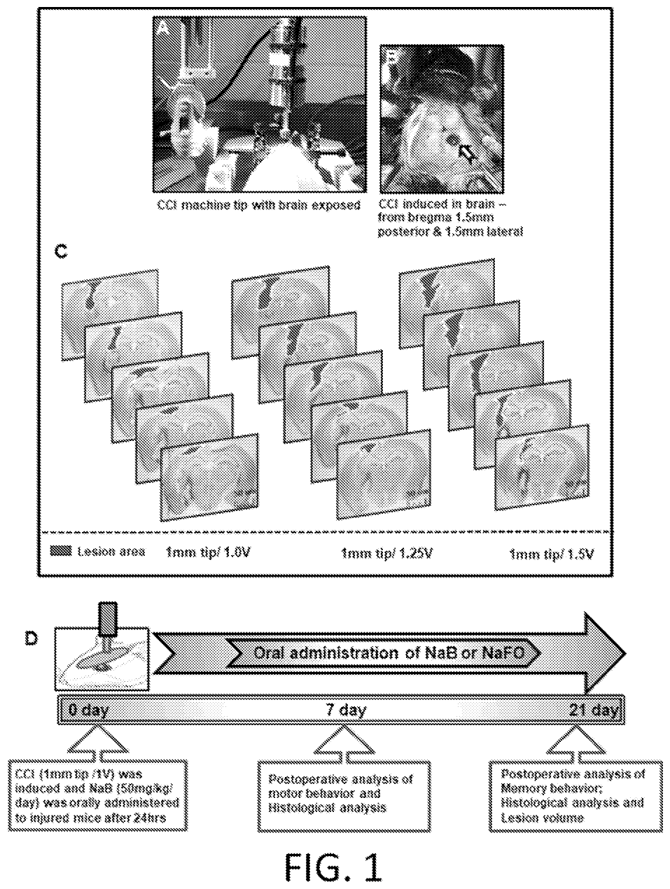

FIGS. 1lA-11H. Restoration of PSD-95, NR2A and GluR1 in the hippocampus of TBI mice by oral administration of GTB. TBI was induced in mice by CCI injury and after 24 h of injury, mice were treated with 50 mg/kg/day of GTB via oral gavage. Twenty-one days after CCI injury, brain sections were double-labeled for NeuN and PSD-95 (FIG. 11A, control; FIG. 111B, CCI; FIG. 11C, CCI+GTB; FIG. 11D, CCI+Vehicle). Results represent analysis of one section of each of six mice per group. Hippocampal tissue extracts from all groups of mice (n=4 per group) were immunoblotted for PSD-95, NR2A and GluR1. FIG. 11E. Actin was run as a loading control. Bands were scanned, and values (Iba1/Actin, FIG. 11F; NR2A/Actin, FIG. 11G; GluR1/Actin, FIG. 11H) presented as relative to control. Data are expressed as mean+SD. Statistical analyses were performed with one way ANOVA.

FIGS. 12A-12G. Effect of GTB on spatial learning and memory in TBI mice. TBI was induced in mice by CCI injury and after 24 h of injury, mice were treated with 50 mg/kg/day of GTB via oral gavage. Twenty-one days after CCI injury, mice were tested by Novel object recognition test (FIG. 12A, Heat map; FIG. 12C, Exploration time), Barnes maze (FIG. 12B, Heat map; FIG. 12D, number of errors; FIG. 12E, latency pr time taken) and T-maze (FIG. 12F, positive turns; FIG. 12G, Negative turns). Six mice were used in each group. Statistical analyses were performed by one way ANOVA followed by Tukey's post hoc test.

FIGS. 13A-13M. GTB treatment recovers motor functions in TBI mice. TBI was induced in mice by CCI injury and after 24 h of injury, mice were treated with 50 mg/kg/day of GTB via oral gavage. Seven days after CCI injury, mice were tested for open-field behavior (FIG. 13A, heat map analysis monitored by using the Noldus system; FIG. 13B, distance moved; FIG. 13C, velocity; FIG. 13D, center frequency; FIG. 13E, rearing), roto rod (FIG. 13F, latency), tail suspension test (FIG. 13G, immobility time), beam walking (FIG. 13H, number of steps; FIG. 131, time taken; FIG. 13J, slips), and grid runway (FIG. 13K, number of steps; FIG. 13L, time taken; FIG. 13M, misplacements). Six mice were used in each group. Statistical analyses were performed by one way ANOVA followed by Tukey's posthoc test.

FIGS. 14A-14M. Effect of GTB on motor functions in TBI mice on 21st day of CCI injury. TBI was induced in mice by CCI injury and after 24 h of injury, mice were treated with 50 mg/kg/day of GTB via oral gavage. Twenty-one days after CCI injury, mice were tested for open-field behavior (FIG. 14A, heat map analysis monitored by using the Noldus system; FIG. 14B, distance moved; FIG. 14C, velocity; FIG. 14D, center frequency; FIG. 14E, rearing), rotorod (FIG. 14F, latency), tail suspension test (FIG. 14G, immobility time), beam walking (FIG. 14H, number of steps; FIG. 141, time taken; FIG. 14J, slips), and grid runway (FIG. 14K, number of steps; FIG. 14L, time taken; FIG. 14M, misplacements). Six mice were used in each group. Statistical analyses were performed by one way ANOVA followed by Tukey's post hoc test.

FIGS. 15A-15L: Effect of sodium benzoate (NaB) on the maturation of oligodendroglial progenitor cells (OPCs) into oligodendrocytes. OPCs were isolated from P1˜P2 neonatal mouse pups, cultured in OPC media for 4 days in vitro (DIV) followed by treatment with 100 μM NaB and sodium formate (NaFO) in the absence of FGF and PDGF.

FIG. 15A) After 18 h of 100 μM NaB treatment in serum-free condition, cells were fixed and then dual immuno-stained with MBP (red) and OPC marker NG2 (green). Nuclei were stained with DAPI (blue). FIG. 15B) Similarly, OPCs were stained with PLP (red) and A2B5 (green) under similar treatment condition. Quantification of MBP+(FIG. 15C), NG2+(FIG. 15D), PLP+(FIG. 15E), and A2B5+(FIG. 15F) cells as a percentage of total cells (DAPI+). Average 5 fields per slide, total 3 slides. Results are mean+SEM. ***p<0.001. FIG. 15G) For protein expression, cells were treated with different doses of NaB for 18 h under serum free condition and then immunoblotted with PLP and MOG. FIG. 15H) Densitometric analyses of the bands relative to beta actin were done for PLP and MOG. ^{a}p<0.001 vs. control PLP; ^{b}p<0.005 vs. control MOG. FIG. 151) Cells were treated with NaB (100 μM) and NaFO (100 μM) for 4 h under serum-free condition followed by monitoring the mRNA expression of myelin-specific genes by real-time PCR (ap<0.01 vs. control PLP; ^{b}p<0.01 vs. control MOG; cp<0.01 vs. control MBP; dp<0.01 vs. control CNPase). OPCs were cultured on the top of randomly-oriented polycaprolactone nanofibers (Nanofiber Solutions; Cat #Z694576) for 7 days. After that, these cells were treated with 100 μM NaB (FIG. 15J) and NaFO (K) for additional 2 days followed by immunofluorescence analysis for MBP (red) (FIG. 15J, control; FIG. 15K, NaB; FIG. 15L, NaFO). Images were displayed in a single red channel as well as merged with phase-contrast image of nanofibers. Representative 3D constructed images of OPCs adhered to nanofibers were also shown at the right side.

FIGS. 16A-16C: Oral NaB stimulates the maturation of OPCs in vivo in the corpus callosum of cuprizone-intoxicated mice. C57/BL6 mice (8-10 week old; male) were fed cuprizone-containing diet (Envigo) for 5 weeks followed by treatment with NaB (50 mg/kg body wt/d) orally via gavage. FIG. 16A) After 3 weeks of treatment with NaB, corpus callosum sections were double-labeled for PLP and A2B5. Mean fluorescence intensity (MFI) of A2B5 (FIG. 16B) and PLP (FIG. 16C) were quantified from one section (two images per section) of each of 5 mice group. Results are mean+SEM of 5 mice per group. ***p<0.001; **p<0.01.

FIGS. 17A-17H: Effect of NaB on myelination in vivo in the corpus callosum of cuprizone-intoxicated mice. C57/BL6 mice (8-10 week old; male; n=5) were fed cuprizone-containing diet (Envigo) for 5 weeks followed by treatment with NaB (50 mg/kg body wt/d) orally via gavage. After 3 weeks of treatment with NaB, corpus callosum sections were immunostained MBP (FIG. 17A) and PLP (FIG. 17B). Mean fluorescence intensity (MFI) of MBP (FIG. 17C) and PLP (FIG. 17D) were quantified from one section (two images per section) of each of 5 mice group. Results are mean+SEM of 5 mice per group. **p<0.01; *p<0.05. FIG. 17E) Corpus callosum sections were stained for luxol fast blue (LFB). FIG. 17F) For electron microscopic studies, 50 μm thick sagittal sections were prepared and stained followed by analysis of corpus callosum sections for different parameters to evaluate axonal ultrastructures. FIG. 17G) G score was calculated in 75 axons per group for all three groups. FIG. 17H) Percentage of myelinated axons was calculated in 7 randomly selected corpus callosum sections of 5 mice per group. ***p<0.001.

FIGS. 18A-18C. Cinnamein inhibits the induction of NO production from LPS- and IFNγ-stimulated mouse RAW 264.7 macrophages. FIG. 18A) Cells preincubated with different concentrations of cinnamein for 6 h were stimulated with 1 μg/ml LPS under serum-free condition. After 24 h of stimulation, the level of nitrite was measured in supernatants by Griess reagent. FIG. 18B) Cells preincubated with 400 μM cinnamein for different hours were stimulated with 1 μg/ml LPS under serum-free condition. After 24 h of stimulation, the level of nitrite was measured in supernatants. FIG. 18C) Cells preincubated with different concentrations of cinnamein for 6 h were stimulated with 25 U/ml IFNγ under serum-free condition. After 24 h of stimulation, the level of nitrite was measured in supernatants. Results are mean+SD of three independent experiments. *p<0.05; **p<0.01; ***p<0.001; NS, not significant.

FIGS. 19A-19B. Cinnamein inhibits LPS- and polyIC-induced production of TNFα in primary mouse microglia. Microglia isolated from 2d old mouse pups were incubated with different concentrations of cinnamein for 6 h followed by stimulation with either 1 μg/ml LPS (FIG. 19A) or 50 μg/ml polyIC (FIG. 19B) under serum-free condition. After 24 h of stimulation, the level of TNFα was measured in supernatants by ELISA. Results are mean+SD of three independent experiments. *p<0.05; **p<0.01; ***p<0.001; NS, not significant.

FIGS. 20A-20B. Cinnamein suppresses the production of IL-1j3 from LPS- and poly IC-stimulated primary mouse microglia. Cells preincubated with different concentrations of cinnamein for 6 h were stimulated with either 1 μg/ml LPS (FIG. 20A) or 50 μg/ml polyIC (FIG. 20B) under serum-free condition. After 24 h of stimulation, the level of IL-10 was measured in supernatants by ELISA. Results are mean+SD of three independent experiments. ***p<0.001.

FIGS. 21A-21B. Cinnamein decreases LPS- and polyIC-induced production of IL-6 in primary mouse microglia. Microglia were incubated with different concentrations of cinnamein for 6 h followed by stimulation with either 1 μg/ml LPS (FIG. 21A) or 50 μg/ml polyIC (FIG. 21B) under serum-free condition. After 24 h of stimulation, the level of IL-6 was measured in supernatants by ELISA. Results are mean+SD of three independent experiments. ***p<0.001.

FIGS. 22A-22B. Cinnamein inhibits the production of proinflammatory cytokines from polyIC-stimulated primary mouse astrocytes. Astrocytes preincubated with different concentrations of cinnamein for 6 h were stimulated with 50 μg/ml poly IC under serum-free condition. After 24 h of stimulation, levels of TNFα (FIG. 22A) and IL-6 (FIG. 22B) were measured in supernatants by ELISA. Results are mean+SD of three independent experiments. *p<0.05; **p<0.01; ***p<0.001.

### DETAILED DESCRIPTION

### A. Definitions

“Nervous system injury,” including central or peripheral nervous system injuries, refers to any injury to the nervous system caused by trauma and/or disease.

The “central nervous system” (CNS) includes the brain, spinal cord, optic, olfactory, and auditory systems. The CNS comprises both neurons and glial cells (neuroglia), which are support cells that aid the function of neurons. Oligodendrocytes, astrocytes, and microglia are glial cells within the CNS. Oligodendrocytes myelinate axons in the CNS, while astrocytes contribute to the blood-brain barrier, which separates the CNS from blood proteins and cells, and perform a number of supportive functions for neurons. Microglial cells serve immune system functions.

“Central nervous system injury” refers to any injury to the central nervous system caused by trauma instead of disease. The term encompasses injuries to the central nervous system that result in loss or impairment of motor function, sensory function, or a combination thereof.

The “peripheral nervous system” (PNS) includes the cranial nerves arising from the brain (other than the optic and olfactory nerves), the spinal nerves arising from the spinal cord, sensory nerve cell bodies, and their processes, i.e., all nervous tissue outside of the CNS. The PNS comprises both neurons and glial cells (neuroglia), which are support cells that aid the function of neurons. Glial cells within the PNS are known as Schwann cells, and serve to myelinate axons by providing a sheath that surrounds the axons.

“Peripheral nervous system injury” refers to any injury to a peripheral nerve caused by trauma instead of disease. The term encompasses all degrees of nerve injury, including the lowest degree of nerve injury in which the nerve remains intact but signaling ability is damaged, known as neurapraxia. The term also includes the second degree in which the axon is damaged but the surrounding connecting tissue remains intact, known as axonotmesis. Finally, the term encompasses the last degree in which both the axon and connective tissue are damaged, known as neurotmesis.

“Traumatic brain injury” or “TBI” refers to an acquired brain injury or head injury in which trauma damages the brain. The damage can be localized, i.e., limited to one area of the brain, or diffuse, affecting one or more areas of the brain.

“Spinal cord injury” means any injury to the spinal cord that is caused by trauma instead of disease. Depending on where the spinal cord and nerve roots are damaged, the symptoms can vary widely, for example from pain to paralysis to incontinence. Spinal cord injuries are described at various levels of “incomplete,” which can vary from having no effect on the subject to a “complete” injury which means a total loss of function. Spinal cord injuries have many causes, but are typically associated with major trauma from motor vehicle accidents, falls, sports injuries, and violence. The abbreviation “SCI” means spinal cord injury.

“Spinal cord contusion” refers to an injury caused by trauma instead of disease in which part of the spinal cord is crushed with part of its tissue spared, particularly the ventral nerve fibers connecting the spinal cord rostral and caudal to the injury.

“Nerve crush injury” refers to traumatic compression of the nerve from a blunt object, such as a bat, surgical clamp or other crushing object that does not result in a complete transection of the nerve.

“Administering” means any method used to deliver the compounds, salts, or compositions to the subject. These include oral routes, intraduodenal routes, parenteral injection (including intravenous, subcutaneous, intraperitoneal, intramuscular, intravascular or infusion), topical, and rectal administration. Those of skill in the art are familiar with administration techniques that can be used, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In some aspects, the compounds and compositions are administered orally.

“Effective amount” refers to a sufficient amount of at least one agent or compound being administered which will relieve or prevent to some extent one or more of the symptoms of the injury being treated. The result can be reduction and/or alleviation of the signs, symptoms, or causes of an injury, or any other desired alteration of a biological system. For example, an “effective amount” for therapeutic uses is the amount of a compound required to provide a clinically significant decrease in the progression or severity of a symptom associated with an injury being treated. An appropriate “effective” amount in any individual case may be determined using techniques such as a dose escalation study.

“Subject” can be any living subject, including mammalian subjects such as a human.

“Prodrug” refers to any pharmaceutically acceptable compound or salt, which, upon administration to the subject, is capable of providing, either directly or indirectly, a benzoic acid salt, e.g., through metabolism in the body.

“Pharmaceutically acceptable” refers to a material, such as a carrier, diluent, or excipient, which does not abrogate the biological activity or properties of the active ingredient, and is relatively nontoxic, i.e., the material may be administered to a subject without causing undesirable biological effects or interacting in a deleterious manner with any of the components of the composition in which it is contained.

“Pharmaceutical composition” refers to a composition comprising a biologically active compound, optionally mixed with at least one pharmaceutically acceptable component, such as carriers, stabilizers, diluents, dispersing agents, suspending agents, thickening agents, or excipients.

### B. Treatment Methods

In one aspect, the method of slowing the progression of or reducing the severity of a symptom associated with a nervous system injury in a subject in need thereof comprises administering to the subject an effective amount of a benzoic acid salt or a prodrug thereof, thereby slowing the progression of or reducing the severity of the symptom associated with the nervous system injury.

In one aspect, the benzoic acid salt, when used, is sodium benzoate, potassium benzoate, calcium benzoate, 2-aminobenzoate, 3-aminobenzoate, 4-aminobenzoate, or any combination thereof.

In one aspect, the prodrug of the benzoic acid salt, when used, is benzyl cinnamate, glyceryl tribenzoate, cinnamic acid, benzyl acetate, benzyl alcohol, benzoic acid, quinic acid, phenylalanine, tyrosine, or any combination thereof.

A variety of nervous system disorders can be treating using the disclosed method. In one aspect, the nervous system injury in the subject is a central nervous system (CNS) injury or a peripheral nerve injury. In a further aspect, the nervous system injury is a spinal cord injury (SCI), spinal cord contusion, or nerve crush injury. For example, when the injury to the nervous system is a spinal cord injury (SCI), the benzoic acid salt or the prodrug thereof can improve nervous system dysfunction caused by trauma to the cervical, thoracic, lumbar or sacral segments of the spinal cord, including without limitation dysfunction caused by trauma to one or more of dermatomes C1, C2, C3, C4, C5, C6, C7, T1, T2, T3, T4, T5, T6, T7, T8, T9, T10, T11, T12, L1, L2, L3, L4 or L5.

In one aspect, the nervous system injury is traumatic brain injury (TBI). In various aspects, TBI can be an injury to the frontal lobe, parietal lobe, occipital lobe, temporal lobe, brain stem, or cerebellum. In some aspects, the TBI is a mild TBI. In a further aspect, the TBI is a moderate to severe TBI. The benzoic acid salts and prodrugs thereof can, in various aspects, cause a detectable improvement in, or a reduction in the progression of, one or more of the following symptoms of TBI: headache, memory problems, attention deficits, mood swings and frustration, fatigue, visual disturbances, memory loss, poor attention or concentration, sleep disturbances, dizziness or loss of balance, irritability, emotional disturbances, feelings of depression, seizures, nausea, loss of smell, sensitivity to light and sounds, mood changes, getting lost or confused, or slowness in thinking.

In another aspect, the nervous system injury is demyelinating disorder. The demyelinating disorder for example can be optic neuritis, X-Adrenoleukodystrophy, Krabbe disease, progressive multifocal leucoencephalopathy, adrenomyeloneuropathy, acute-disseminated encephalomyelitis, acute haemorrhagic leucoencephalitis, multiple sclerosis, Balo's disease (concentric sclerosis), Charcot-Marie-Tooth disease, Guillain-Barre syndrome, HTLV-I associated myelopathy, neuromyelitis optica (Devic's disease), Schilder's disease, transverse myelitis, or a combination thereof.

In general, the injuries that can be treated with the disclosed method can result in a number of symptoms which can be alleviated, slowed, or prevented using the benzoic acid salt or the prodrug thereof. In one aspect, administering the effective amount of the benzoic acid salt or the prodrug thereof results in a reduction of glial inflammation, improvement in motor function or coordination, or an improvement in learning or memory dysfunction. In a further aspect, particularly when the injury being treated is an injury to the CNS, administering the effective amount of the benzoic acid salt or the prodrug thereof prevents or reduces the severity of a symptom associated with mental depression. One non-limiting example of a symptom of mental depression is the level of physical activity the subject is motivated to engage in.

In one aspect, it can be useful to administer the benzoic acid salts or prodrugs thereof before a nervous system injury has significantly progressed. For example, the effective amount of the benzoic acid salt or the prodrug thereof can be administered within 24 hours after the nervous system injury, e.g., within 23, 22, 21, 20, 19, 18, 17, 16, 15, 14, 13, 12, 11, 10, 9, 8, 7, 6, 5, 4, 3, 2, or 1 hour after the nervous system injury. In another aspect, the effective amount of the benzoic acid salt or the prodrug thereof can be administered 24 hours or longer after the nervous system injury. In a further aspect, the effective amount of the benzoic acid salt or the prodrug thereof can be administered within 24 hours after the nervous system injury, and administration of the benzoic acid salt or the prodrug thereof can continue for a period of time, e.g., days, weeks, months, or years after injury.

In one aspect, the subject has been diagnosed with the nervous system injury prior to the administering step. In a further aspect, the subject has been diagnosed with a central nervous system (CNS) injury or a peripheral nerve injury prior to the administering step. In a further aspect, the subject has been diagnosed with a spinal cord injury (SCI), spinal cord contusion, or nerve crush injury prior to the administering step. In a still further aspect, the subject has been diagnosed with traumatic brain injury (TBI) prior to the administering step.

In one aspect, prior to the administering step, the subject being treated has not been diagnosed with or treated for a urea cycle disorder, glycine encephalopathy, multiple sclerosis, Parkinson's disease, Alzheimer's disease, Huntington disease, or an autism spectrum disorder. Examples of autism spectrum disorders include Asperger's syndrome, childhood disintegrative disorder, and pervasive developmental disorder.

In one aspect, the subject being treated is older than 12 years old. In a further aspect, the subject being treated is at least 18 years old. In a still further aspect, the subject being treated is at least 21 years old. In other aspects, the subject can be any age, including younger than 12 years old.

In one aspect, the benzoic acid salt or the prodrug thereof can be administered as a pharmaceutical composition comprising the benzoic acid salt or the prodrug thereof and a pharmaceutically acceptable excipient, with the composition comprising greater than 0.10% of the benzoic acid salt or the prodrug thereof by weight of the composition, e.g., greater than 0.5%, greater than 1%, greater than 2%, greater than 5%, greater than 10%, greater than 15%, greater than 20%, greater than 30%, greater than 40%, or greater than 50% by weight of the benzoic acid salt or the prodrug thereof, up to 99% by weight of the benzoic acid salt or the prodrug thereof, based on the total weight of the pharmaceutical composition. For instance, the pharmaceutical composition can comprise benzoic acid or the prodrug thereof in an amount ranging from 1.10% to 50% or more, by weight of the composition.

In a further aspect, the pharmaceutical composition can comprise only the benzoic acid salt or the prodrug thereof as the active ingredient for treating the injury. In other words, in one aspect, the benzoic acid salt or the prodrug thereof can serve as the only active ingredient. In a further aspect, the pharmaceutical composition of the present disclosures comprises an active pharmaceutical ingredient that consists essentially of, or in other aspects, consists of, the benzoic acid salt or the prodrug thereof.

In one aspect, the total daily dose of the benzoic acid salt or the prodrug thereof is 100 mg/day, 120 mg/day, 150 mg/day, 180 mg/day, 200 mg/day, 225 mg/day, 250 mg/day, 300 mg/day, 400 mg/day, 500 mg/day, 1000 mg/day, 1500 mg/day, 2000 mg/day, 2500 mg/day, 3000 mg/day, 3500 mg/day, or 4000 mg/day, administered to the subject as a single or multiple doses depending on various factors such as the route of administration. In a further aspect, the total daily dose of the benzoic acid salt or the prodrug thereof is 1000 mg/day to 4000 mg/day, e.g., 1000 mg/day to 3000 mg/day, or 1000 mg/day to 2000 mg/day.

In one specific aspect, these total daily doses of the benzoic acid salt or the prodrug thereof can be administered orally as a single or multiple doses. In one aspect, the composition administered to the subject can be formulated in a form suitable for oral administration. For example, the composition can be formulated in a form of dry powder, a tablet, a lozenge, a capsule, granule, or a pill. The pharmaceutically acceptable excipient includes, but is not limited to, a filler, a binder, a preservative, a disintegrating agent, a lubricant, a suspending agent, a wetting agent, a solvent, a surfactant, an acid, a flavoring agent, polyethylene glycol (PEG), alkylene glycol, sebacic acid, dimethyl sulfoxide, an alcohol, or any combination thereof.

### C. Examples

The following examples further illustrate this disclosure. The scope of the disclosure and claims is not limited by the scope of the following examples.

### 1. Materials and Methods

### a. Animals

Male C57BL6 mice (7-8 weeks old) purchased from Harlan, Indianapolis, IN were used for this study. Animal maintenance and surgical procedure were conducted in compliance with NIH guidelines for the Care and Use Committee and were approved by the Rush University Animal Care and Use Committee. Animals were housed in an environment with stable temperature and 12h light-dark cycle. Water and food were provided ad libitum.

### b. Controlled Cortical Impact Procedure

To induce brain injury in mice, the controlled cortical impact (CCI) injury technique was applied. Adult C57BL6 mice were anesthetized with 2% isoflurane and allowed to breathe normally without tracheal intubation. Body temperature was maintained at 37° C. on a heating pad and monitored by a rectal probe during the surgery. The depth of anesthesia was observed by a gentle toe pinch without causing any injury. The heads of anesthetized mice were shaved with sterile electric shaver and skin was cleaned with betadine solutions. Then, the animal's head was fixed in stereotaxic frame and TBI was induced by using the CCI technique (FIG. 1A-D). Initially, a midline skin incision was performed to expose the skull and 4 mm diameter craniotomy was made in the right side of exposed skull with the coordinates −1.5 mm AP and −1.5 mm ML using the stereotaxic apparatus. Then the brain was exposed in this burr-hole with intact dura. Under surgical microscope control, the Leica Impact One Stereotaxic Impactor (Leica Mi-crosystems, Buffalo Grove, IL) attached with 1.0 mm rounded metal tip was angled vertically towards the brain surface with intact dura. Subsequently, a mild injury was unilaterally induced with a strike velocity of 1.0 m/s in the right side of exposed brain region. A sterile sponge immobilization board was used to support below the head to act as a support like cushion during the induction of brain injury. After impact injury, the damage was produced in the cerebral cortex, causing extensive structural damage in the surrounding region. Sham group animals underwent the similar surgical procedure but without CCI injury. Then, the operated animal was removed from the stereotaxic holder and the skin incision was lightly sutured to close the incised region. All operated animals were placed in a thermal blanket for the maintenance of body temperature within the normal limits. These animals were monitored until the recovery from anesthesia and over the next three consecutive postoperative days.

Using small laboratory animals, like mice, for producing a clinically related TBI model is a challenging task in TBI research. The effect of TBI may vary in physical and psychological outcomes depending on the extent of damage to the brain. Some symptoms may appear immediately after the injury, while others may appear days or weeks later. Therefore, a fixed 1 mm rounded tip with different velocity was used for the standardization purpose. In this study, mice were randomly divided into three groups and CCI was applied with a 1 mm rounded tip with three velocities, viz. 1.0V, 1.25V and 1.50V for the induction of mild, moderate and severe injuries, respectively (FIG. 1C). At the end of one week post-operative period, all three groups of animals were perfused with 4% paraformaldehyde to remove the brain. Subsequently, the brain sections were made at 40 μm thickness. Using cresyl violet staining, the histopathological features of brain damage that revealed prominent tissue damage in cortex and hippocampus region in the mild injury group were studied. However, no noticeable damage was seen in the contralateral side of the brain in this group of mice. In moderate injury group, more damage was found in tissues in the ipsilateral cortex and hippocampus region of mice brain and recovery of mice after surgery was found extremely slow and fatal in some cases. Further, serious tissue damage was noticed in both cortex and hippocampus of the ipsilateral side of brain after surgery in severe injury group (FIG. 1C). Recovery of mice was minimal and the injury produced became fatal in many cases in this group of mice. Therefore, based on the histopathological observations of three types of injury groups, the mild type of CCI injury (1 mm tip and 1.0V) was selected to delineate the beneficial effects of cinnamon metabolite NaB in the improvement of cognitive and motor functions after brain injury (FIG. 1C).

### c. Treatment with Sodium Benzoate or Sodium Formate

Sodium benzoate (“NaB”) and sodium formate (“NaFO”) were solubilized in 0.1% methyl cellulose solution. Starting from 24 hours of CCI injury, mice were orally treated with NaB or NaFO (50 mg/kg/day) once daily for 7 postoperative days. Later, the oral treatment was continued every alternate days till 21 postoperative days and following behavior analysis the mice were sacrificed for histological and biochemical studies.

### d. Experimental groups and NaB/NaFO Treatment

FIG. 1D shows the experimental design used in this study. All mice were randomized into the following groups:

Group 1: Control/Sham group (n=6 per group): Mice underwent surgery without any in-jury and treatment.

Group 2: CCI group (n=6 per group): Mice underwent CCI injury and no treatment was carried out.

Group 3: CCI+NaB treatment (n=6 per group): Mice were subjected to CCI and NaB (50 mg/kg/day) treatment orally was started 24 hours after the induction of injury.

Group 4: CCI+NaFO treatment (n=6 per group). Mice were subjected to brain injury and NaFO (50 mg/kg/day) treatment orally was started 24 hours after the induction of injury.

### e. Western Blotting

Western blotting was performed as described in earlier studies. Equal amount of proteins were electrophoresed in 10% or 12% SDS-PAGE and transferred onto nitrocellulose membrane. The blot was probed with primary antibodies overnight at 4° C. The following are the primary antibodies used in this study and are detailed in Table 1 below: anti-iNOS (1:1000, BD Bio-sciences), anti-Iba1 (1:1000, Abcam), anti-GFAP (1:1000, Santa Cruz Biotechnology, Dallas, TX), and anti-R-actin (1:5000, Abcam). Following the overnight incubation, primary antibodies were removed and the blots were washed with phosphate buffer saline containing 0.1% Tween-20 (PBST) and corresponding infrared fluorophore tagged secondary antibodies (1:10,000, Jackson Immuno-Research) were added at room temperature. The blots were then incubated with secondary antibodies for 1 hour. Later, blots were scanned with an Odyssey infrared scanner (Li—COR, Lincoln, NE). Band intensities were quantified using the ImageJ software (NIH, USA).

TABLE 1

| | | | | Application/ |
|---|---|---|---|---|
| Antibody | Manufacturer | Catalog | Host | Dilution |
| GFAP | Dako | Z0334 | Rabbit | IF/1:2000 |
| iNOS | BD | 610432 | Mouse | IF/1:500 |
| | Biosciences | | | |
| Iba1 | Abcam | ab5076 | Goat | IF/1:500 |
| GFAP | Dako | Z0334 | Rabbit | WB/1:1000 |
| iNOS | BD | 610432 | Mouse | WB/1:1000 |
| | Biosciences | | | |
| Iba1 | Abcam | ab5076 | Goat | WB/1:1000 |
| Actin | Abcam | ab1801 | Mouse | WB/1:5000 |

### f. Immunohistochemistry

Mice were anesthetized with ketamine-xylazine mix solutions and perfused with PBS and then with 4% paraformaldehyde (w/v) in PBS, followed by dissection of the brain for immunofluorescence microscopic examination. Briefly, the dissected brains were incubated in 10% sucrose for 3 hours and then followed by 30% sucrose overnight at 4° C. Then the brains were embedded in optimal cutting temperature medium (Tissue Tech) at −80° C. and processed for conventional cryosectioning. Frozen sections (40 μm thickness) were treated with cold ethanol (−20° C.), washed with PBS, blocked with 2% BSA in PBST, and double labeled with two primary antibodies (Table-1). After three washes with PBST, sections were incubated with Cy2 and Cy5 (Jackson ImmunoResearch Laboratories). The sections were mounted and observed under an Olympus IX81 fluorescence microscope. Counting analysis was performed using Olympus Microsuite V software with the help of a touch counting module.

### g. Quantification of Lesion Volume Using Stereological Techniques

The estimation of lesion volume was performed based on the Cavalieri method of unbiased stereology using the StereoInvestigator software (MicroBright Biosciences, USA). Both the ipsilateral and contralateral hemisphere of brain volumes were determined using the Cavalieri estimator with a 1 mm grid spacing 1 mm. Every fourth section was analyzed beginning from a random start point. Lesion volume was estimated by subtracting the volume of the ipsilateral hemisphere from that of the contralateral hemi-sphere. Then the volume of lesion cavity estimated in brain section of untreated mice was compared with lesion volume of brain sections of drug treated mice.

### h. Behavioral analysis

Analysis of behaviors in animals were conducted on the 7th and 21st postoperative days after CCI injury. These time-points for behavioral testing were selected based upon earlier studies with these animal models where behavioral abnormalities were seen at these time points.

### i. Open field behavior

The performance of animals in open field test was analyzed as described in earlier studies. Briefly, each animal was allowed to move freely to explore an open field arena designed with a square shaped wooden floor measuring 40×40 cm, with walls 30 cm high for 5 min. A video computer 6 (Basler Gen I Cam—Basler acA 1300-60) connected to a Noldus computer system was fixed in top facing-down on the open field arena. Each mouse was placed individually on center of the arena and the performance was monitored by the live video tracking system. The central area was arbitrarily defined as a square of 20×20 cm (half of the total area).

### j. Rotarod

The forehindlimb motor coordination and balance in animals was observed using the rotarod test as described in earlier studies. Briefly, each mouse was placed on the confined section of the rod and trial was initiated with a smooth increase in speed from 4 rpm to 40 rpm for 5 mins. If the mouse did not fall from the rod, it was removed from the rod after 5 mins. The latency to fall was measured in seconds and used for the analysis. Following the CCI injury, each mouse performed the task three trials during the testing sessions and the average score on these three trials was used as the individual rotarod score. Each trial on the rod was terminated when the mice fell off the rod or held on to the rod by hanging and completed improper revolutions.

### k. Tail suspension test

Mice were subjected to the tail suspension test using a methodology as described in earlier studies. The mice were gently hung upside down by the tail using the non-toxic adhesive tape 50 cm above the floor for 6 mins. Immobility time was defined as the period of time during which the mice only hung passively, without any active movements. An increased immobility time is defined as a depression-like behavior.

### l. Nesting behavior

A nestlet consisting of a 5 cm×5 cm pressed cotton square was kept inside the cage be-tween 5 pm. and 6 pm. Next morning between 9 am. to 10 am, two observers blind to the experimental procedures scored the quality of nest built by the mice using a 5-point scale as follows: Score 1 (>90% of nestlet intact), Score 2 (50% to 90% of nestlet intact), Score 3 (10% to 50% of nestlet intact but no recognizable nest site), Score 4 (<10% of nestlet intact, nest is recognizable but flat), Score 5 (<10% of the nestlet intact, nest is recognizable with walls higher than the mouse body).

### m. Beam runway

The beam runway was made of smooth wooden material and measured 65 cm length×0.7 cm breadth×4 cm height. A black box with an opening was fixed at one end and an aversive stimulus (bright lamp) at the other end of beam. This test was used to evaluate the complex coordination and balance of mice while traversing the beam and we performed the procedure as described in earlier studies. The mouse was placed on the beam near the light source and the light was turned ‘on’. This makes the animal move into the box to avoid the aversive stimulus, which was then turned off. Six repetitions were performed with a 2 mins resting period inside the box. The parameters measured were the time taken (sec) to reach the box and the number of steps with contralateral limb drag/slips. An error was considered whenever the paw slipping on the beam and the number of slips were counted. The beam walk analysis was performed by an observer blinded to the treatment at 7th and 21st postoperative day.

### n. Grid Runway

The grid runway (65 cm length×8 cm breadth×1 cm intervals) made of parallel grid bars with interbar intervals of 1 cm apart and grid were kept above the surface on a table during the testing session. The soft padding was positioned under the grid run-way in the event for protection to avoid serious injury, if the animal falls from the grid. Each mouse was allowed to walk freely on grid and the time taken and number of steps to cross the runway was noted. Each successful foot placement on grid was recorded as a step. However, an error was considered whenever the paw slips through the grid or the paw misses a bar and extends downwards through the plane of bars. The locomotor behavior of animal on grid was evaluated by an observer blinded to the treatment on 7th and 21st day after CCI injury.

### o. Barnes Maze Test

The Barnes maze test was performed as described in our earlier studies [44, 49, 58]. Briefly, the mice were initially trained for 2 consecutive days followed by examination on day 3. After each training session, maze and escape tunnel were thoroughly cleaned with a mild detergent to avoid instinctive odor avoidance due to mouse's odor from the familiar object. On day 3, a video camera (Basler Gen I Cam—Basler acA 1300-60) connected to a Noldus computer system was placed above the maze and was illuminated with high voltage light that generated enough light and heat to motivate animals to enter into the escape tunnel. The performance was monitored by the video tracking system (Noldus System). Cognitive behavior parameters were examined by measuring latency (duration before all four paws were on the floor of the escape box) and errors (incorrect response before all four paws were on the floor of the escape box).

### p. T-maze

Mice were initially habituated in the T-maze for 2 days under food-deprived conditions. Food reward was provided for at least 5 times over a 10 mins period of training. T-maze was cleaned with mild detergent solution between each testing session, so as to minimize the animal's ability to use any olfactory clues. The food-reward side was always associated with a visual cue. Each time the animal consumed food-reward and it was considered as a positive turn.

### q. Novel Object Recognition (NOR) Test

This test evaluates the animal's ability to recognize the novel object in the environment and monitor short-term memory. Initially, the mice were placed in a square novel box (20 in. long×8 in. high) surrounded with an infrared sensor. Two plastic toys (2.5-3 in. size) that varied in color, shape, and texture were placed in specific locations in the environment 18in. away from each other. The mice were able to freely explore the environment and objects for 15 mins and were then placed back into their individual home cages. After 30 min intervals, the mice were placed back into the environment, with the 2 objects in the same locations, but now one of the familiar objects was replaced with a third novel object. The mice were again allowed to freely explore both objects for 15 min. The familiar and novel objects were thoroughly cleaned with a mild detergent after each testing session.

### r. Statistical Analysis

Based on previous studies of similar type and complexity, six mice are expected to give >80% power for all behavioral experiments. Statistical analyses were performed with Student's t-test for two-group comparison and One-way ANOVA followed Tukey's multiple comparison tests as appropriate for multiple comparison by using GraphPad Prism 7. Data are represented as mean±SD or mean±SEM as stated figure legends. Statistical significance was determined at the level of p<0.05.

### 2. NaB Treatment Attenuates Glial Activation in CCI-Induced TBI Mice

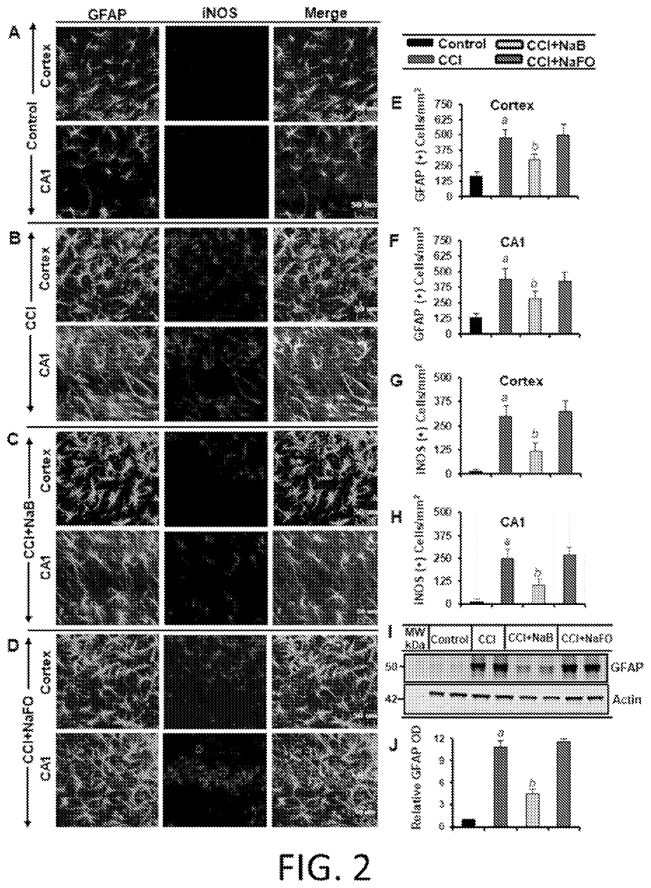

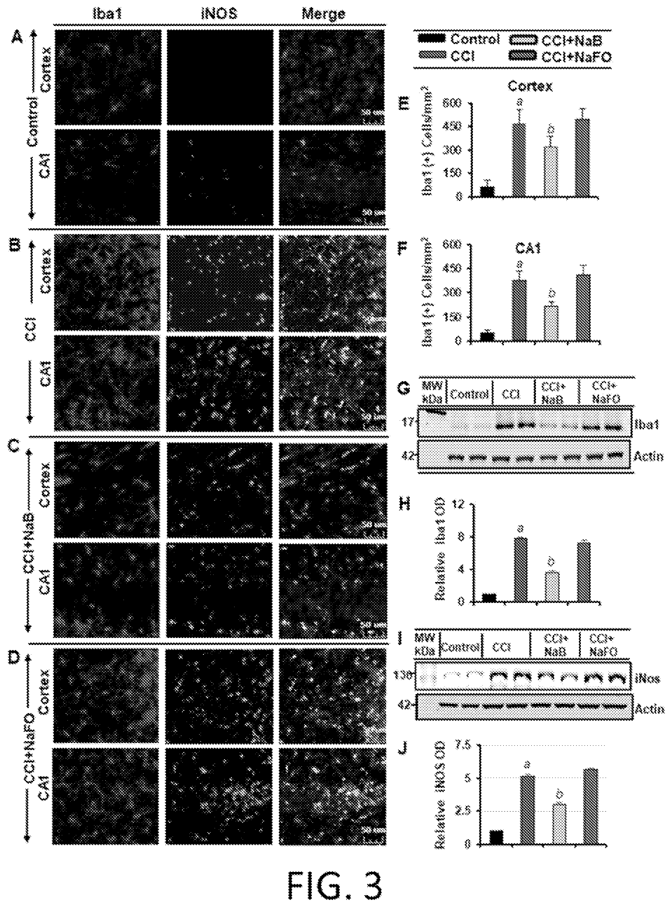

Recent findings have established microglial and astroglial activation and associated neuroinflammation as important pathological events in different neuroinflammatory and neurodegenerative disorders, including brain injury. Immediately after the initial CCI injury, tissue environment modifies to activate glial cells. Accordingly, following CCI insult (FIG. 1), a marked increase in the number of GFAP-positive astrocytes (FIG. 2A, FIG. 2B, FIG. 2E, and FIG. 2F) and Iba1-positive microglia (FIG. 3A, FIG. 3B, FIG. 3E, and FIG. 3F) in cortex and hippocampus region of mice on day 7 post-injury was observed as compared to sham control. Western blot analysis of hippocampal extracts also corroborated this increase in GFAP (FIG. 2I and FIG. 2J) and Iba1 (FIG. 3I and FIG. 3J). However, oral treatment of CCI-insulted TBI mice with NaB led to decrease in both GFAP-positive astrocytes (FIG. 2A-2F) and Iba1-positive microglia (FIG. 3A-3F). This result was specific as sodium formate (NaFO) remained unable to inhibit glial activation in the hippocampus of TBI nice (FIG. 2A, FIG. 2B, FIG. 2E, and FIG. 2F and FIG. 3A, FIG. 3B, FIG. 3E, and FIG. 3F).

Decrease and/or normalization of protein levels of GFAP (FIG. 2I-2J) and Iba1 (FIG. 3I-3J) in the hippocampus of NaB-treated TBI mice is also evident from Western blots. Activated glial cells are known to express inducible nitric oxide synthase (iNOS) that produce excessive nitric oxide to cause nitrosative stress in a neuroinflammatory milieu. Correspondingly, the level of iNOS was higher in cortex and hippocampus of TBI mice on day 7 post-injury in comparison to sham control (FIG. 2 and FIG. 3). Double-label immunofluorescence analysis revealed that increased iNOS was present in both GFAP-expressing astrocytes (FIG. 2A-2H) and Iba1-positive microglia (FIG. 3A-3F). However, treatment of TBI mice with NaB, but not NaFO, led to inhibition of iNOS in both cortex and hippocampus (FIG. 2C-2D and FIG. 3C-3D). These findings were confirmed by quantitative analyses (FIG. 2G-2H) and Western blot (FIG. 3I-3J). Collectively, these results denote that NaB is capable of reducing the glial inflammation in vivo in the CNS of CCI-induced TBI mice.

### 3. Oral NaB Stimulated Remyelination in Mice with TBI

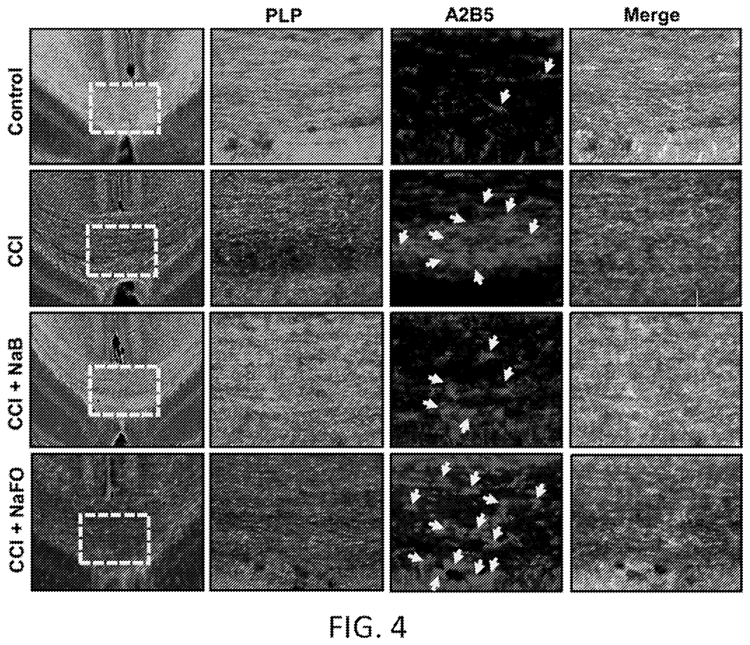

Proteolipid protein (PLP) is a marker of oligodendrocytes and A2B5 is a marker of oligodendroglial progenitor cells (OPC). After 21 days of treatment, brain sections were double-labeled with antibodies against PLP and A2B5. The images in FIG. 4 show that, as expected, the level of PLP was very low in the corpus callosum of mice with TBI and many A2B5 positive OPCs were localized in the demyelinated area. However, NaB treatment markedly increased the level of PLP in corpus callosum of mice with TBI. Accordingly, NaB treatment also decreased the number of OPCs in the corpus callosum of TBI mice. These results were specific to NaB, as NaFO, a molecule structurally similar to NaB without the benzene ring, remained unable to restore the level of PLP and decrease the number of OPCs in the corpus callosum of TBI mice.

### 4. NaB Treatment Reduced the Lesion Volume in CCI-Induced Mice

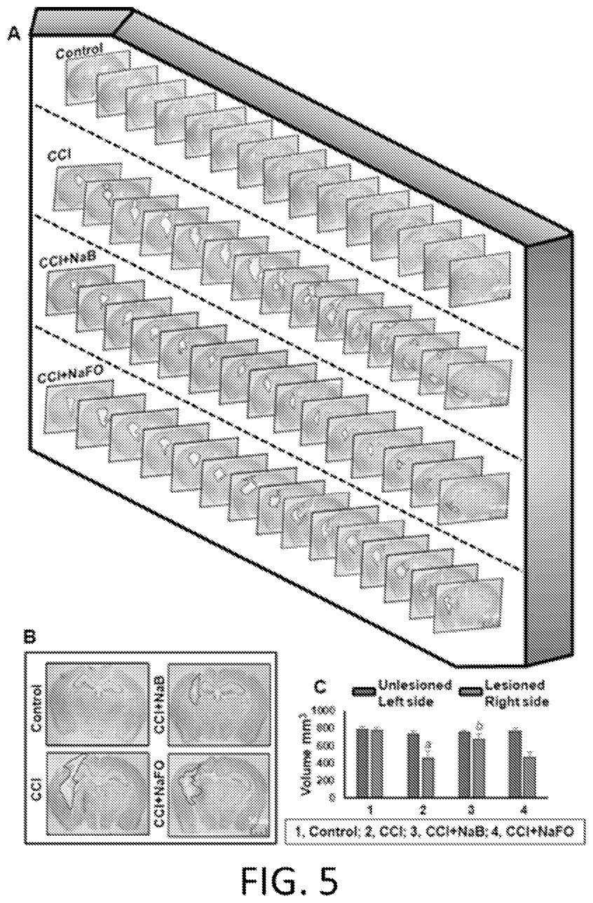

Since oral NaB reduced glial inflammation in the CNS of TBI mice, next, it was examined whether NaB treatment was capable of reducing lesion volume. Therefore, lesion volume was measured in cresyl-violet stained sections and compared between untreated and treated groups. FIG. 5A shows cresyl violet stained brain sections arranged serially to evidence the volume of lesion cavity from different groups of mice. After 21 days post-injury, typical lesion was observed, including the enlarged cavity, originating from cortex through hippocampus and connecting to lateral ventricle in CCI-induced TBI mice as compared to none in sham control (FIG. 5B). On the other hand, oral administration of NaB, but not NaFO, reduced the size of lesion cavity in CCI-induced mice. Quantitative analysis of lesion volume using the Cavalieri Stereological techniques revealed that total lesion volume in the whole hemisphere was significantly reduced after oral treatment of NaB when compared to either untreated or NaFO-treated TBI-mice (FIG. 5C).

### 5. Oral NaB Improved Open Field Behaviors and Locomotor Activities in Mice with TBI

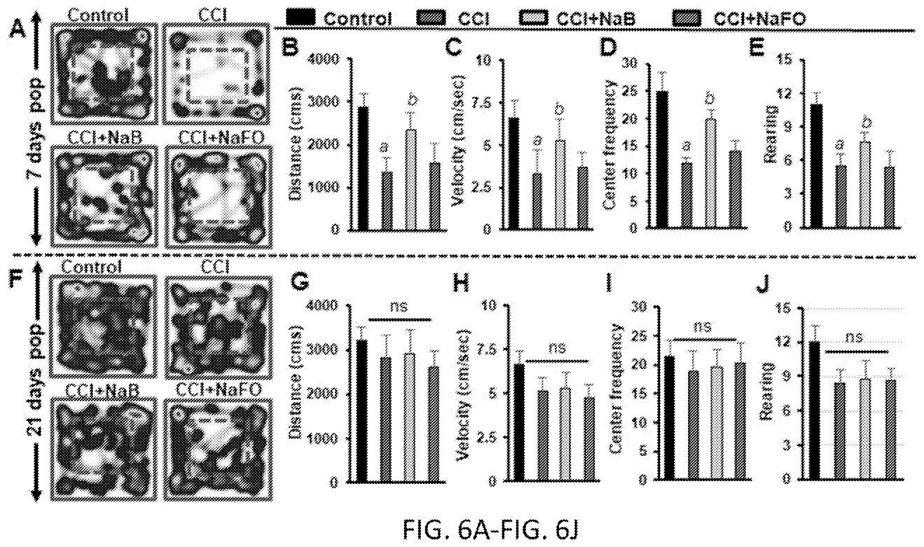

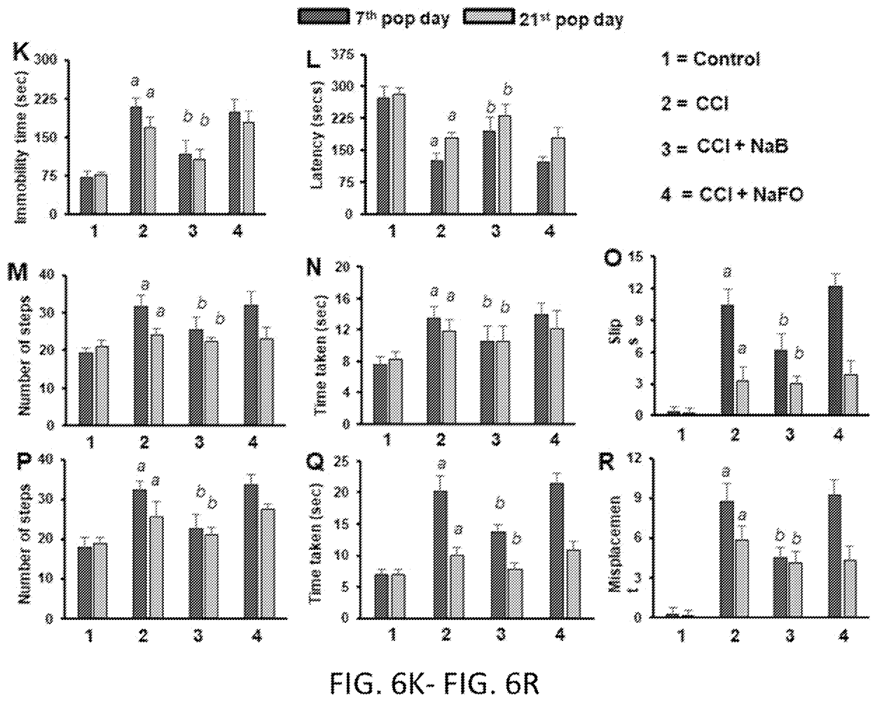

The foremost therapeutic objective of neuroprotection research is to limit secondary tissue loss and to preserve or improve the behavioral functions. Therefore, to analyze whether oral administration of NaB protected not only the organizational damage but also functional shortages caused by CCI insult, the overall gait activities were examined. A video camera 6 (Basler Gen I Cam—Basler acA 1300-60) connected to a Noldus computer system remained stationary on top facing-down on the open-field arena for recording general locomotor behaviors. FIG. 6A and FIG. 6F represent heat maps summarizing the overall activity of mice in the open field test at 7 day and 21 day post-injury, respectively. As compared to either untreated or NaFO-treated TBI mice, the general locomotor activity showed a significant improvement in NaB treated TBI-mice at 7 day post-injury (FIG. 6A-6E). Functional upgrading was clearly visible from distance traveled (FIG. 6B), velocity (FIG. 6C), center frequency (FIG. 6D) and rearing behavior (FIG. 6E). On the other hand, significant differences in overall movements were not observed between treated and untreated TBI-groups at 21 day post-injury (FIG. 6).

Subsequently, the recovery of motor coordination and balance activity in all group of CCI-insulted mice using the rotarod test at 7 day and 21 day post-injury was also examined. Following CCI injury, mice without treatment showed a significant decrease in latency to fall at 7 day post-injury and this motor activity remained impaired on rotarod throughout the 21 days post-injury as compared to sham-control group. However, treatment of CCI-injured mice with NaB, but not NaFO, resulted in prolonged latencies by maintaining the proper body movements and balancing functions on the rotarod test (FIG. 6L).

Depression is a common symptoms noticed during the initial stage of brain injury. Therefore, depression-like behavior in CCI-injured mice was monitored. Previous studies in TBI research have demonstrated that depression in mice can be analyzed by an increase in duration of immobility. Hence, this test to was performed to examine the neuroprotective effect of NaB on depression like behavior in CCI-insulted mice. At 7 days post-injury, CCI-mice without any treatment showed significantly longer immobility time than sham controls (FIG. 6K). On the other hand, CCI-mice treated with NaB exhibited significantly less immobility time compared to either untreated or NaFO-treated ones. Upon NaB treatment, the duration of immobility was close to the normal level. These results suggest that NaB is capable of controlling the depression-like behavior in CCI-insulted mice.

TBI-induced damage always impairs the connection between brain and muscles, ultimately affecting gait movements. Consequently, gait-related impairments in CCI-mice on beam and grid were analyzed as these two multifaceted runways appeared to divulge different patterns of movement than the ones on the open-field behavior test. Earlier studies have revealed that these beam and grid runways are particularly useful in models of unilateral TBI because it allows scientists the opportunity to analyze and compare the contralateral-versus-ipsilateral limb movement. Hence, the neuroprotective role of NaB on recovery of gait functions in the unilateral CCI model using beam and grid runways was examined. CCI-mice had a tendency to drag the contralateral pelvic limb while walking. This type of behavior was not seen in sham controls. Further, sham controls did not show significant changes in the latency or number of foot-steps to cross the beam after surgery.

However, none of the CCI-mice were able to cross the beam on the day of surgery and the day after surgery (FIG. 6M-6O). On the 7 day post-injury, CCI-mice without treatments showed significant deficits to balance the body on the beam or paw slipping through the grid. CCI-mice without treatments showed poor performance in gait behavior exhibiting more latency, steps and foot-fault, or foot misplacement while crossing the beam as compared to sham controls. Similar results were seen for grid analysis (FIG. 6P-6R). However, upon treatment with NaB, but not NaFO, CCI-injured mice demonstrated significant improvement in gait movement at beam and grid runways.

NaB-treated CCI mice also exhibited significant upgrade in latency, foot-steps, foot-slips, and foot-misplacement as compared to either untreated or NaFO-treated CCI mice (FIG. 6M-6R). On the other hand, at 21 day post-injury, CCI-mice recovered considerably to the near normal-level as we did not see significant changes in these parameters with respect to sham controls. As a result, NaB treatment also did not display significant protection at either beam walking or grid runways of CCI-mice at 21 days post-injury.

### 6. Oral NaB Protected Spacial Learning and Memory in Mice with TBI

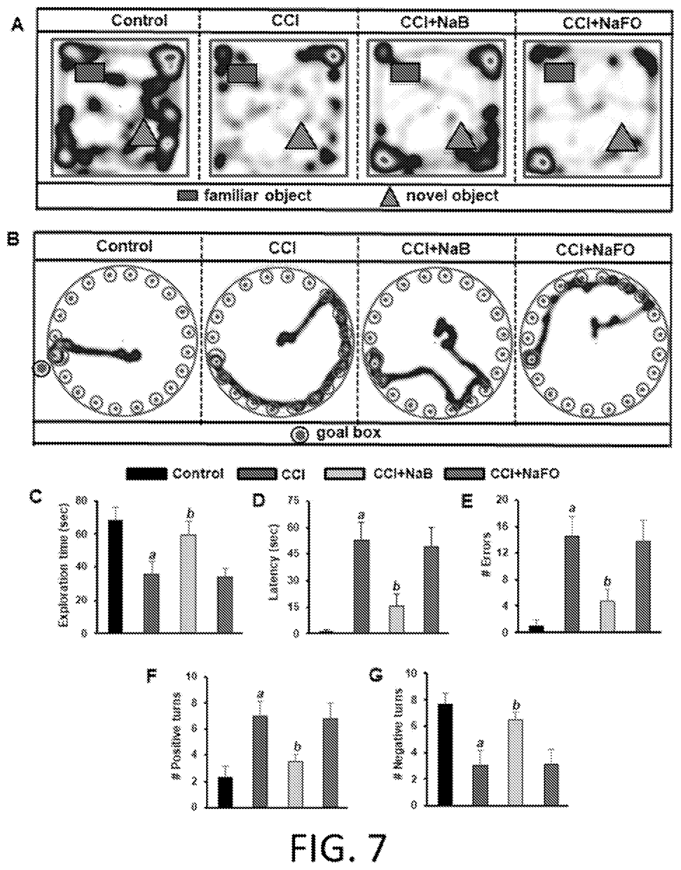

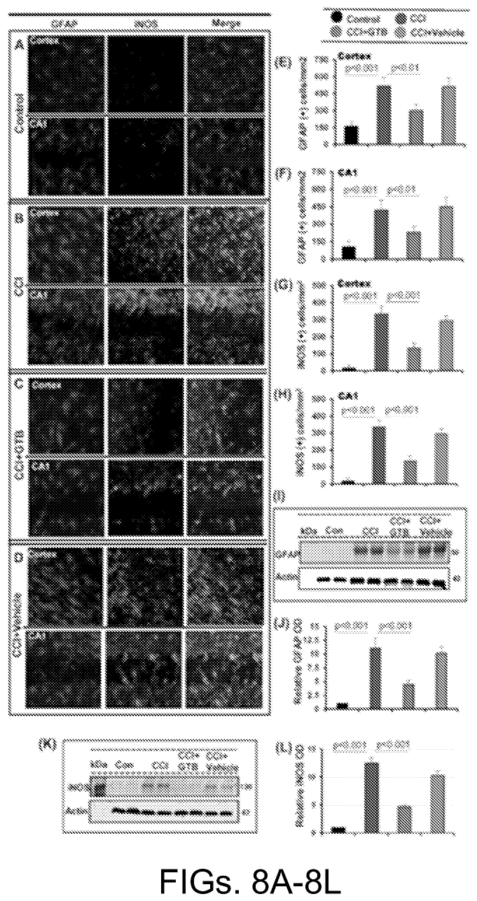

TBI survivors often suffer from problems with learning and memory throughout the rest of their lives. Therefore, to examine whether oral NaB protects memory and cognitive function in TBI mice, mouse performance on novel object recognition (NOR), Barnes maze, and T maze masks was monitored. FIG. 7A shows heat maps demonstrating the novel object recognition of mice after 21 days of treatment. FIG. 7C shows the exploration time results for this same test.

The Barnes circular maze test is a hippocampus-dependent cognitive task which requires spatial reference memory. FIG. 7B shows heat maps demonstrating the Barnes circular test results of TBI mice after 21 days of treatment, and FIG. 7D shows the latency time, and FIG. 7E shows the number of errors made. TBI mice did not find the reward hole easily, required more time (latency), and made more errors. On the other hand, NaB-treated TBI mice were as capable as healthy control mice in finding the target hole with less latency and fewer errors.

Similar results were found in the T maze test. FIG. 5F shows the number of positive turns, and FIG. 5G shows the number of negative turns made during this test after 21 days of treatment. TBI mice displayed fewer number of positive turns and a higher number of negative turns than the sham control. Once again, NaB treatment significantly improved the hippocampus dependent memory performance in TBI mice as exhibited by a higher number of positive turns and a lower number of negative turns.

These results were specific to NaB. NaFO, a negative control of NaB, remained unable to improve hippocampus-dependent behaviors in TBI mice.

### 7. Discussion

Although TBI is a major cause of death and disability in US, despite intense investigation, no effective treatment is available until today to improve the quality of life in patients with TBI except for regular medical evaluation and care. Therefore, describing a safe and effective therapy to modulate the pathological process of TBI, resulting in improvement in behavioral outcome is an important area of research. Several pieces of evidence outlined in this study clearly support the conclusion that NaB is capable of suppressing the disease process of TBI in a CCI-induced mouse model. While the TBI caused a massive lesion cavity, oral NaB treatment started from 24 h after the CCI decreased the lesion volume and restored the structural-tissue integrity of damaged hippocampus. In contrast, treatment with NaFO, a NaB analog without the benzene ring, remained unable to exhibit such protection. NaB treatment also reduced the depression-like behavior, attenuated motor dysfunction and enhanced cognitive performance in mice with TBI. Furthermore, consistent to its safety track record, oral NaB did not cause any side effects (for example, decrease in body weight, loss of hair, fecal boli, infection, untoward behavior, etc.). These results suggest that oral NaB may be beneficial for treatment of TBI and that NaB should not be toxic for TBI patients.

Glial activation and upregulation of proinflammatory molecules in the CNS participate in the pathogenesis of a number of neurodegenerative diseases including TBI. It is known that immediately after TBI, microglia and astroglia in the brain are activated to produce proinflammatory cytokines (e.g. IL-10, TNFα, etc.), proinflammatory enzymes (e.g. inducible nitric oxide synthase or iNOS), reactive oxygen species, etc., in toxic amounts for a prolonged time period to ultimately cause axonal damage. Here, it has been demonstrated that NaB treatment reduces the level of microglial marker Iba1 and astroglial marker GFAP and decreases the expression of iNOS in the hippocampus of mice with TBI. Therefore, although NaB treatment started from 24 h after TBI in a therapeutic mode, it is capable of reducing and/or normalizing glial inflammation in TBI mice.

The signaling mechanisms by which glial cells are activated are poorly understood. It is reported that NaB inhibits LPS-induced expression of iNOS and proinflammatory cytokines in microglia. TLR4 is a prototype receptor for LPS. However, NaB has no effect on the level of TLR4 in LPS-stimulated microglia, indicating that NaB deters LPS-induced expression of proinflammatory molecules without involving its receptor TLR4. Interestingly, intermediates (HMG-CoA, mevalonate and famesyl pyrophosphate), but not the end products (cholesterol and coenzyme Q), of the mevalonate pathway reverse the anti-inflammatory effect of NaB in microglia. Suppression of LPS-induced activation of NF-κB and expression of iNOS in glial cells by farnesyltransferase inhibitor proposes an important role of farnesylation reaction in the upregulation of iNOS gene. Consistent to a role of farnesylation in the activation of p21ras, it is seen that p21ras signaling plays an important role in the expression of proinflammatory molecules in glial cells. Therefore, suppression of p21ras activation in microglial cells by NaB indicates that NaB attenuates glial inflammation via suppression of p21ras activation.

Until now, no effective interdictive therapy is available for stopping the progression of TBI. Although anticoagulants are there to prevent blood clots and improve blood flow, anti-anxiety medications for reducing fear and nervousness, antidepressants to treat symptoms of depression and mood instability, anticonvulsants for preventing seizures, muscle relaxants to decrease muscle spasms, except anticoagulants others are peripheral treatments. Moreover, some of these medications show limited symptomatic relief exhibiting a number of side effects. On the other hand, there are several advantages of NaB over available TBI therapies. First, NaB is objectively safe. It is water soluble and if consumed in excess, it is secreted through the urine. Second, NaB can be taken orally, the least painful route of drug treatment. Oral NaB reduced glial activation in vivo in the hippocampus and improved cognitive performance in TBI mice. Third, NaB is economical compared to the other existing anti-TBI therapies. Fourth, entry of drugs through the blood-brain barrier (BBB) is an important issue for the treatment of CNS disorders. Although in the early phase of TBI, the BBB remains compromised, with time, the integrity of BBB improves and therefore, BBB-permeable drugs will be helpful for neuroprotection in TBI patients. NaB has also been detected in the brain of mice that were treated with cinnamon orally. Therefore, after oral treatment, NaB enters into the brain.

### 8. Protection of Mice from Controlled Cortical Impact Injury By Food Additive Glyceryl Tribenzoate

Here, we examined the neuroprotective effect of GTB in controlled cortical impact (CCI) mouse model of TBI. We demonstrate that after oral administration GTB is capable of attenuating glial activation, reducing the level of pro-inflammatory molecules, decreasing lesion volume, and improving synaptic structure in CCI-induced TBI mice. Functionally, oral GTB restored locomotor performance and improved learning and memory in TBI mice, high-lighting possible therapeutic application of GTB in TBI.

### 9. Attenuation of Astroglial and Microglial Activation in CCI-Induced TBI Mice by Oral GTB

Astrocytes and microglia are two important cell types of the central nervous system. However, studies over the last three decades have revealed that upon activation, these cells release different proinflammatory molecules to participate in the pathogenesis of different neuroinflammatory and neurodegenerative disorders, including TBI.

Therefore, we examined the effect of oral GTB on glial activation in the CNS of TBI mice. At first, we monitored astroglial activation and as expected, CCI insult induced astroglial activation in cortex and hippocampus as revealed by enhanced GFAP expression on day 7 post-injury as compared to sham control (FIG. 8A-B). This finding was corroborated by counting of GFAP positive cells in both cortex (FIG. 8E) and hippocampus (FIG. 8F).

Increase in GFAP following TBI was further confirmed by Western blot analysis of hippocampal extracts (FIG. 8I-J). Recently we have seen that oral administration of GTB at a dose of 50 mg/kg body wt/d alleviates Huntington pathology in mice and inhibits the adoptive transfer of experimental allergic encephalomyelitis (EAE), an animal model of multiple sclerosis (MS), in mice. Therefore, here, CCI-insulted mice were treated with GTB orally via gavage at a dose of 50 mg/kg body wt/d and we observed decrease in GFAP positive astro cytes (FIG. 8A-F) and the level of GFAP protein (FIG. 8I-J) in the hippocampus of TBI mice upon GTB treatment. This result was specific as we did not find such change with vehicle treatment (FIG. 8A-F & I-J). Activated astro cytes express different proinflammatory molecules including inducible nitric oxide synthase (iNOS), which is known to produce excessive nitric oxide to cause nitrosative stress in a neuroinflammatory milieu.

Therefore, we examined the status of iNOS in the hippocampus and cortex of GTB-treated and untreated TBI mice. As expected, we also found increase in iNOS-positive cells (FIGS. 8A, B, G, & H) and the level of iNOS protein (FIG. 8K-L) in the brain of TBI mice as compared to sham control. Many GFAP-positive astrocytes colocalized with iNOS (FIG. 8A-D). However, similar to the suppression of astroglial activation, oral GTB also decreased iNOS-positive cells (FIGS. 8A, B, G, & H) and the level of iNOS protein (FIG. 8K-L) in the brain of TBI mice.

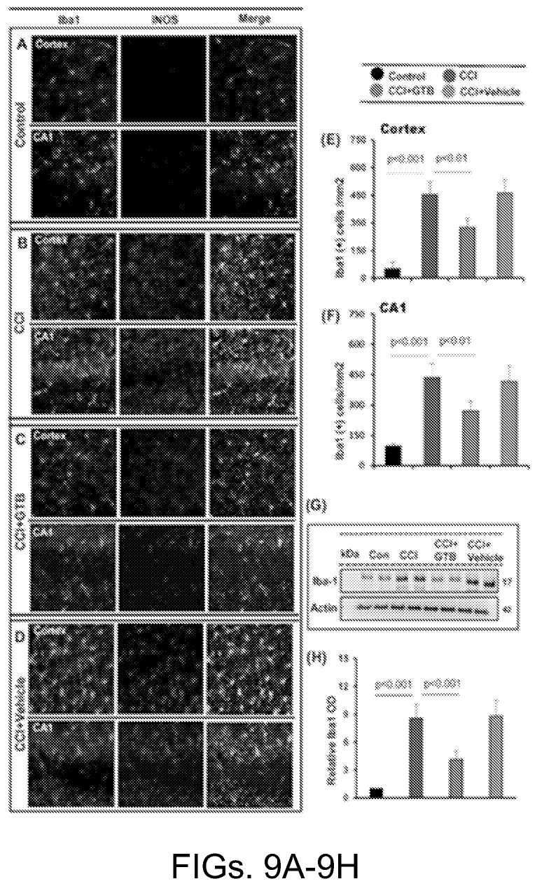

Next, we investigated microglial activation and found marked increase in Iba1-posi-tive microglia in cortex and hippocampus of TBI mice as compared to sham control (FIGS. 9A, B, E, & F).

This result was confirmed by Western blot of Iba1 in hippocampal extracts (FIG. 9G-H). Double labeling experiment also showed colocalization of Iba1-positive microglia with iNOS (FIG. 9A-D). However, similar to the attenuation of astroglial activation, oral adminis-tration of GTB, but not vehicle, reduced the number of Iba1-positive astrocytes (FIG. 9A-F) and the level of Iba1 protein (FIG. 9G-H) in the brain of TBI mice. Together, these results suggest that oral GTB is capable of decreasing both astroglial and microglial activation in the hippocampus of TBI mice.

### 10. Oral Administration of GTB Reduces the Lesion Volume in the CCI Model of TBI

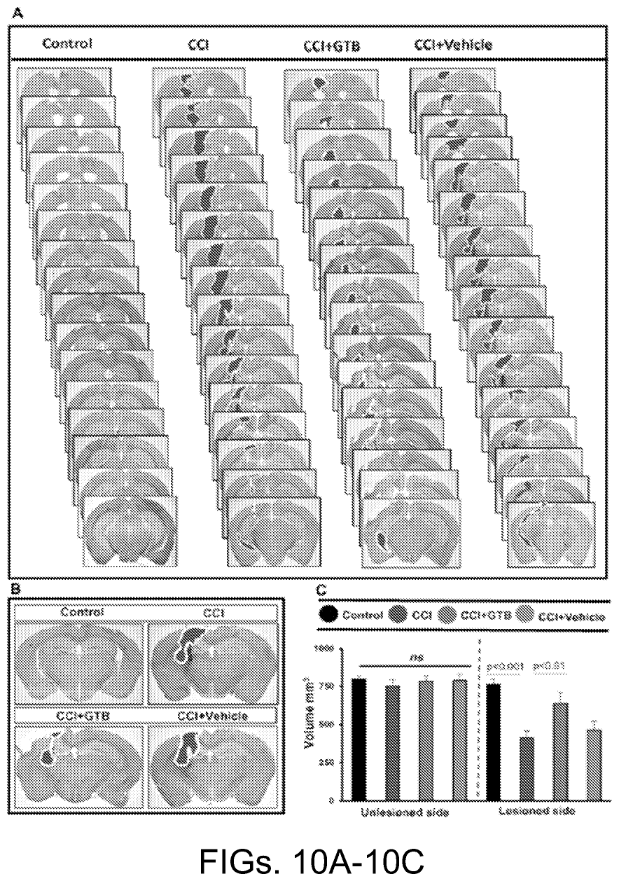

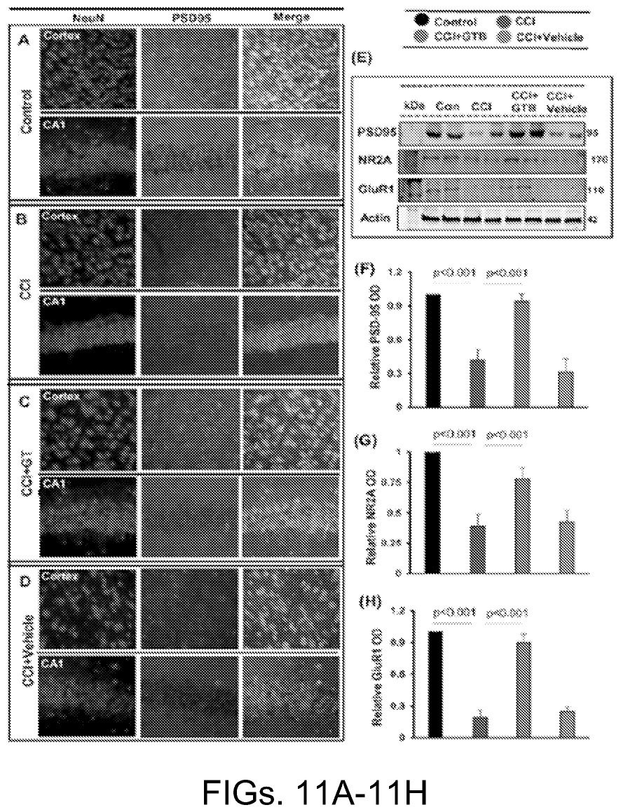

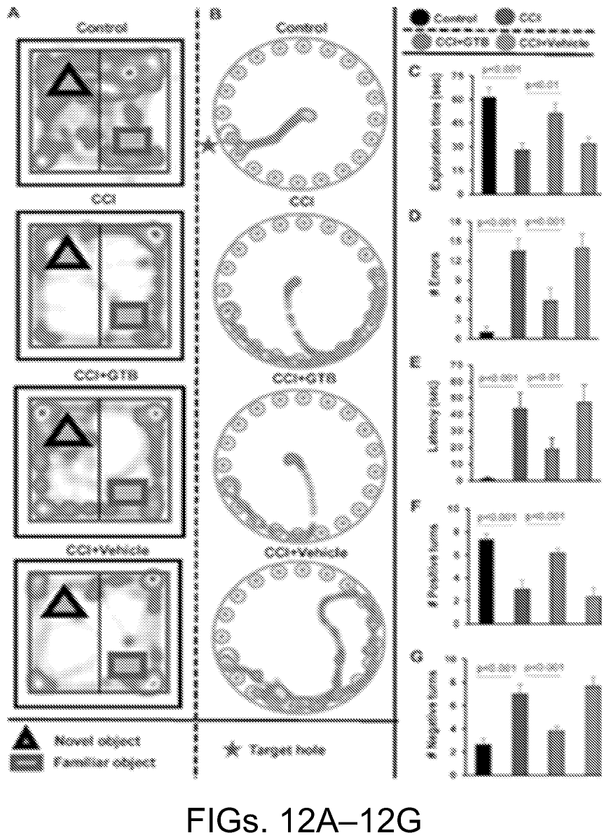

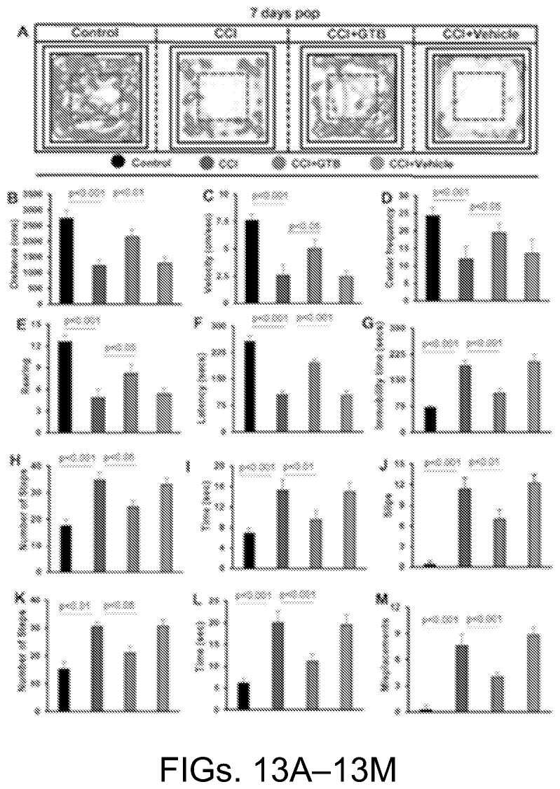

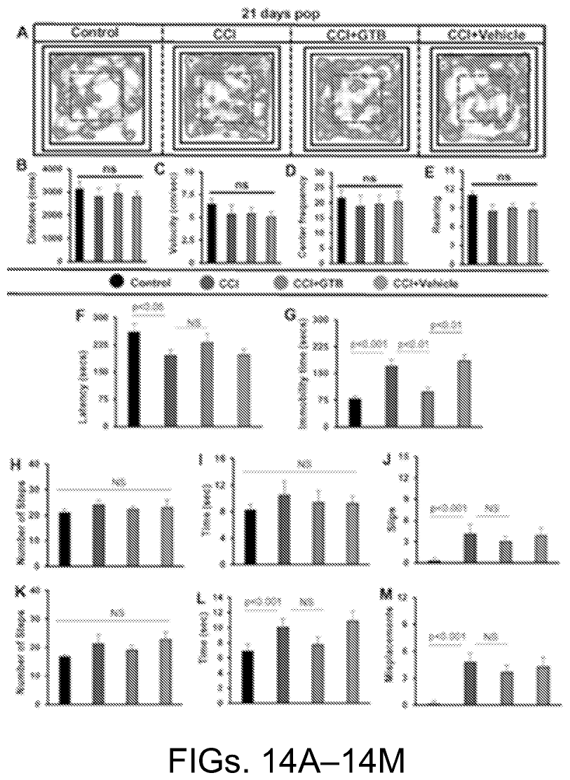

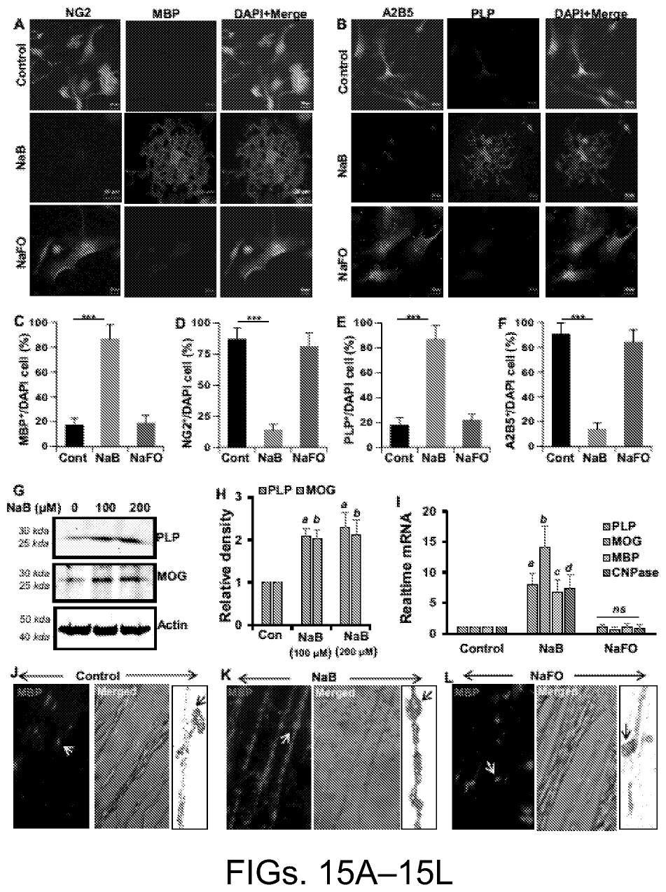

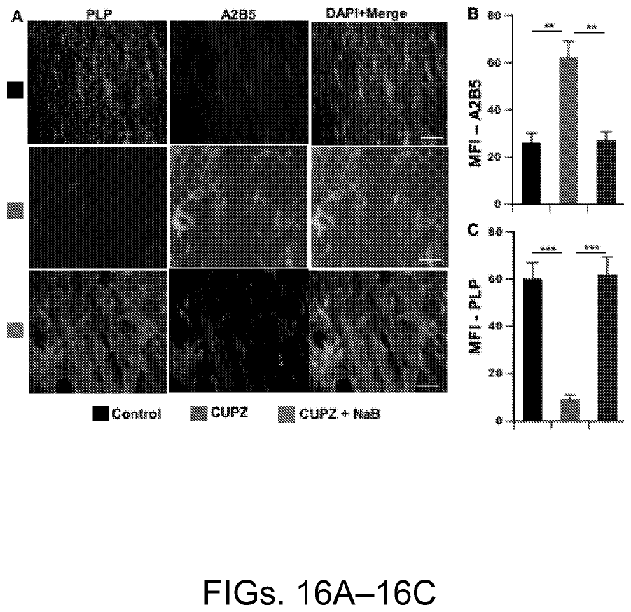

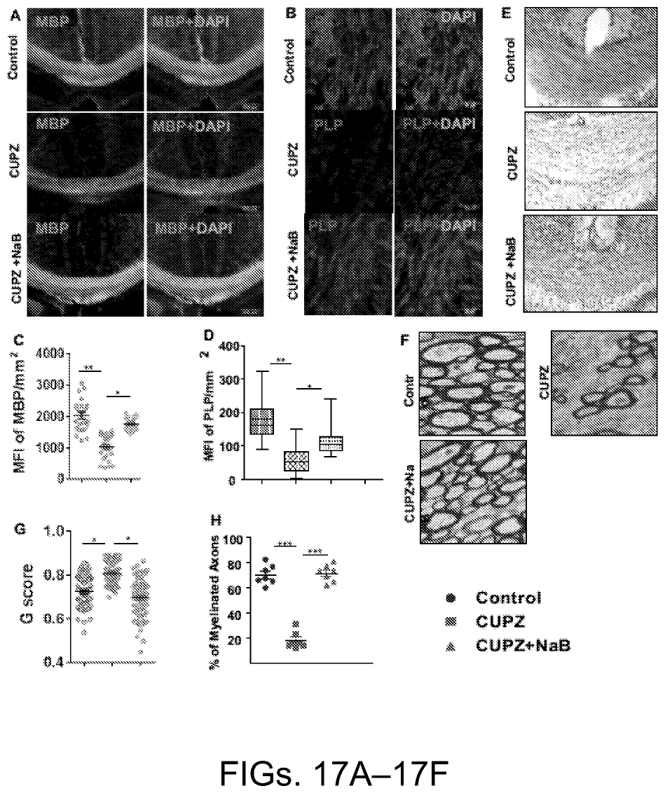

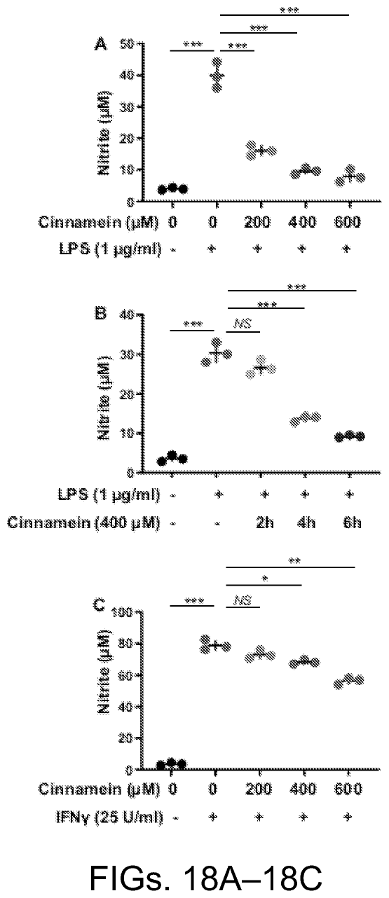

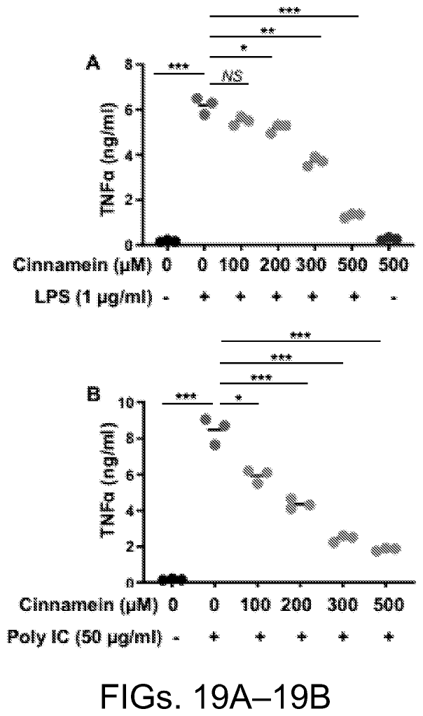

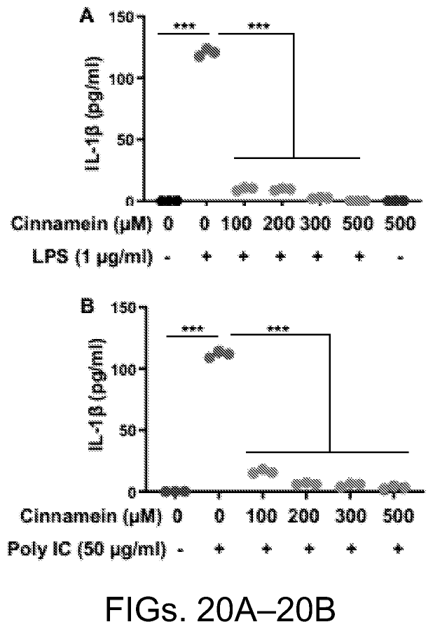

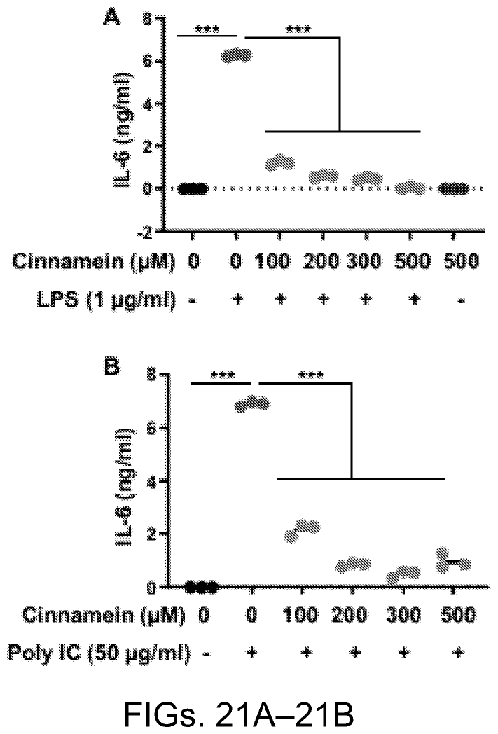

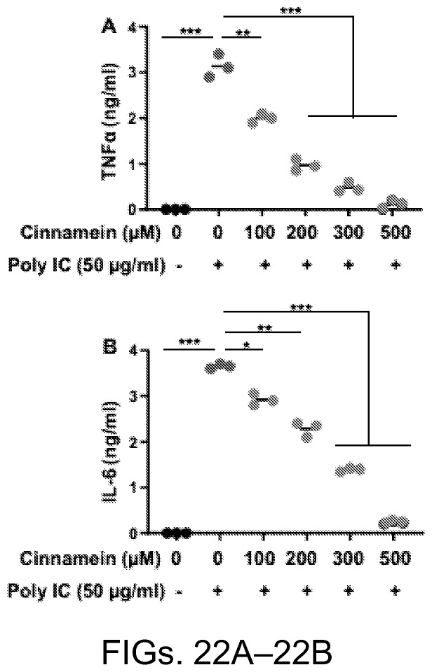

Since GTB treatment inhibited astroglial and microglial activation in the brain of TBI mice, next, we decided to monitor whether oral GTB could reduce the lesion volume after 21 days post-injury. For measuring lesion volume, brain sections were stained with hematoxylin and eosin (H&E). FIG. 10A displays H&E-stained brain sections arranged serially to show the volume of lesion cavity from different groups of mice. As anticipated, we found typical lesion with the distended cavity, originating from cortex through hippocampus and involving to the lateral ventricle in TBI mice as compared to no lesion in sham control (FIG. 10B). However, consistent to the suppression of astroglial and microglial inflammation, treatment with GTB, but not vehicle, reduced the size of lesion cavity in TBI mice (FIG. 10A-B). This was also corroborated by quantitative analysis of lesion volume using the Cavalieri Stereological techniques, which revealed the decrease in total lesion volume in the whole hemisphere upon GTB treatment as com-pared to either un-treated or vehicle-treated TBI mice (FIG. 10C).

### 11. GTB Treatment Resto-Res Synapse Maturation in the Brain of CCI-Insulted Mice

Recent studies have shown that TBI has a major impact on synapse structure and function via a combination of the instant mechanical insult and the resultant secondary injury processes (e.g. inflammation), ultimately leading to synapse loss. For example, according to Witcher et al, TBI causes chronic cortical inflammation mediated by activated microglia, ultimately leading to synaptic dysfunction.

Therefore, since GTB treatment reduces glial inflammation, we examined whether GTB could protect the synapse in TBI mice. PSD-95 is involved in synapse development and maturation. Double labeling of brain sections for NeuN and PSD-95 indicated loss of synaptic maturation in cortex and hippocampus of TBI mice as indicated by decrease in PSD-95 after 21 days post-injury in comparison to sham control mice (FIG. 11A-B). On the other hand, we did not observe such loss of NeuN in cortex and hippocampus of TBI mice (FIG. 11A-B). Western blot analysis of hippocampal tissues also confirmed a marked decrease in PSD-95 in the hippocampus of TBI mice as compared to sham mice (FIG. 11E-F). However, consistent to the suppression of astroglial and microglial inflammation, treatment with GTB, but not vehicle, upregulated the level of PSD-95 in the brain of TBI mice (FIG. 11A-F).

In addition to PSD-95, other molecules such as NR2A and GluR1 are also involved in synapse maturation. Therefore, we also monitored the levels of NR2A and GluR1 and found significant decrease in both NR2A (FIGS. 11E & G) and GluR1 (FIGS. 11E & H) in the hippocampus of TBI mice after 21 days post-injury in comparison to sham control mice. Similar to the upregulation and/or restoration of PSD-95, GTB treatment increased the level of NR2A (FIGS. 11E & G) and GluR1 (FIGS. 11E & H) in the hippocampus of TBI mice. These results were specific as we did not observe any such increase in NR2A and GluR1 by vehicle treatment (FIGS. 11E, G & H). These results suggest that oral GTB is capable of restoring synapse maturation in the hippocampus of TBI mice.

### 12. Oral GTB Protects Cognitive Functions in TBI Mice

Many TBI survivors suffer from cognitive deficits throughout the rest of their lives. It has been reported that impaired synaptic alterations are implicated in contributing to cognitive defects in TBI. Since GTB treatment protected and/or improved synapse development and maturation in hippocampus and cortex of TBI mice, we examined whether GTB could protect cognitive functions in TBI mice after 21 days post-injury. While to monitor short term memory, we employed novel object recognition (NOR) test, for spatial learning and memory, mouse behaviors were analyzed on Barnes maze and T-maze.

As evident from NOR task, TBI mice spent less time with novel object as compared to sham control mice (FIGS. 12A & C). On the other hand, upon treatment with GTB, but not vehicle, TBI mice spent significantly more time with novel object (FIGS. 12A & C), indicating improvement in short term memory by oral GTB. Barnes maze is a hippocampus-dependent memory task that requires spatial reference memory. It showed that TBI mice without treatments did not find the reward hole easily (FIG. 12B), made more errors (FIG. 12D) and required greater time (latency) (FIG. 12E) as compared to sham control mice. However, GTB-treated, but not vehicle-treated, TBI mice performed much better on Barnes maze (FIG. 12B), made less errors (FIG. 12D), and took less time (FIG. 12E) to find the target hole as compared to untreated TBI mice. In T-maze as well, TBI mice without treatments exhibited less number of positive turns (FIG. 12F) and greater number of negative turns (FIG. 12G) than sham control mice. Consistent to NOR task and Barnes maze, oral administration of GTB, but not vehicle, considerably enhanced the hippocampus-dependent memory performance in TBI mice as exhibited by a higher number of positive turns (FIG. 12F) and a lower number of negative turns (FIG. 12G) than untreated TBI mice.

### 13. GTB Treatment Improves Locomotor Functions in TBI Mice after 7 Days of CCI Injury

The principal therapeutic aim of TBI research is to preserve or recover the behavioral functions. Since GTB treatment protected cognitive functions in TBI mice, next, we investigated whether GTB also protected overall locomotor activities. For recording general locomotor behaviors, we employed the Noldus computer system connected to a video camera 6 (Basler Gen I Cam—Basler acA 1300-60) that remained stationary on top facing down on the open field arena. FIG. 13A represents heat maps summarizing the overall movement of mice in the open field arena after 7 day of CCI injury.

As expected, TBI mice exhibited decreased open field activity in comparison to sham control with respect to heat map (FIG. 13A), distance travelled (FIG. 13B), velocity (FIG. 13C), center frequency (FIG. 13D), and rearing (FIG. 13E) on 7th day post CCI injury. However, treatment of TBI mice with GTB, but not vehicle, led to significant increase in open-field behavior (FIG. 13A-E).

Next, we used rotorod test to examine motor coordination and balance activity of mice. Similar to open field activity, TBI mice exhibited significant decrease in latency to fall at 7 day post CCI injury as compared to sham control (FIG. 13F). On the other hand, oral administration of GTB, but not vehicle, improved rotorod performance as seen by increase in latency (FIG. 13F).

Depression is a noticeable symptom of TBI particularly during the initial stage of brain injury, which can be monitored in mice by tail suspension test. Therefore, we performed this test to monitor the effect of GTB treatment on depression like behavior in TBI mice. As evident from FIG. 13G, TBI mice on 7th day of CCI insult exhibited significantly higher immobility time than sham control, indicating more depressive behavior in TBI mice than sham mice. However, GTB-treated TBI mice displayed significantly less immobility time during tail suspension test than either untreated or vehicle-treated TBI mice (FIG. 13G), suggesting inhibition of depressive behavior by GTB.

TBI is known to damage the connection between brain and muscles, thereby impairing gait movements. Therefore, we employed beam walking to monitor gait behavior and observed poor gait movement of TBI mice as compared to sham control (FIG. 13H-J). TBI mice used more steps (FIG. 13H), took more time (FIG. 131) and made more slips (FIG. 13J) than sham control mice while crossing the beam. However, oral administration of GTB, but not vehicle, improved beam walking of TBI mice (FIG. 13H-J). To further confirm the results, we also used grid runway that allows scientists the opportunity to analyze and compare gait activities.

Similar to that found with beam walking, TBI mice also performed poorly in comparison to sham control on grid runway in terms of number of steps (FIG. 13K), time taken (FIG. 13L) and misplacement (FIG. 13M). In this case as well, GTB treatment improved the performance of TBI mice on grid runway (FIG. 13K-M). Together, these results indicate improved locomotor performance of TBI mice on 7th day of CCI injury upon GTB treatment.

On the other hand, many of the locomotor parameters improved spontaneously on 21st day of CCI injury and we also did not observe any significant change after GTB treatment (FIG. 14A-M). For example, no significant change was seen in all parameters tested for open-field behavior (FIG. 14A, heat map; FIG. 14B, distance traveled; FIG. 14C, velocity; FIG. 14D, center frequency; FIG. 14E, rearing) and some parameters tested for beam walking (FIG. 14H, number of steps; FIG. 141, time taken) and grid runway (FIG. 14K, number of steps). Only on tail suspension test, significant impairment was seen in TBI mice as compared to untreated TBI mice and GTB treatment also led to significantly less immobility time during tail suspension test than either untreated or vehicle-treated TBI mice on 21st day of CCI injury (FIG. 14G), suggesting that GTB can inhibit depressive behavior even in the later phase of TBI.

### 14. Conclusions

In summary, we have demonstrated that oral GTB, a flavoring ingredient, reduces glial activation, decreases lesion cavity, and protects cognitive and motor behaviors in a preclinical model of TBI. Our results decipher an important neuroprotective effect of GTB, suggesting that GTB may be repurposed for therapeutic intervention in TBI.

### 15. Sodium Benzoate (NaB) Stimulates the Maturation of Oligodendroglial Progenitor Cells (OPCs) into Oligodendrocytes

Downregulation of myelin proteins and subsequent loss of myelin sheath are considered to be pathological features of multiple sclerosis as well as neurological conditions such as traumatic brain injury. Therefore, we wanted to explore the effect of NaB on remyelination. Oligodendrocytes are generated from OPCs as a result of a reduction of precursor markers such as NG2 and A2B5 with subsequent induction of myelinating proteins such as myelin basic protein (MBP) and proteolipid protein (PLP). Interestingly, we found that NaB stimulated the differentiation of OPCs into oligodendrocytes (FIGS. 15A-F). On the other hand, NaFO, a structural analog of NaB, did not promote the maturation of OPCs to oligodendrocytes (FIGS. 15A-F), indicating the specificity of OPC maturation effect of NaB. Accordingly, NaB treatment increased the level of PLP and myelin oligodendrocyte glycoprotein (MOG) in OPCs (FIGS. 15G-H). These results were corroborated by mRNA analysis of MBP, PLP, MOG, and CNPase (FIG. 151). To understand the functional significance of this finding, we examined the effect of NaB on myelination of synthetic fibers and found stimulation of myelination by NaB, but not NaFO (FIGS. 15J-L).

### 16. Effect of NaB on the Remyelination of Corpus Callosum in Cuprizone-Intoxicated Mouse Model of Demyelination

Next, we examined the effect of NaB on the remyelination in vivo in mouse brain corpus callosum. Corpus callosum is a region, which is primarily affected in different inflammatory demyelinating diseases including MS. As expected, we found decrease in myelin protein PLP and increase in OPC marker A2B5 in the corpus callosum of cuprizone-intoxicated mice as compared to control mice (FIGS. 16A-C). However, NaB treatment increased the level of PLP and decreased the level of A2B5 (FIGS. 16A-C), suggesting that NaB can promote remyelination in the corpus callosum of cuprizone-intoxicated mice. Since, MBP is the marker of myelin integrity, we stained corpus callosum sections with MBP and found loss of MBP in cuprizone-intoxicated mice that increased after NaB treatment (FIGS. 17A & C). Similar results were found in case of PLP, another stability marker of myelin fibers (FIGS. 17B & D). These results were confirmed by LFB staining (FIG. 17E) as well as ultrastructural details by electron microscopy (FIGS. 17F-H).

### 17. Cinnamein: An Anti-Inflammatory Agent

Chronic inflammation driven by macrophages, microglia and astrocytes plays an important role in the pathogenesis of several autoimmune, inflammatory as well as neurodegenerative disorders. Upon activation, macrophages, microglia and astrocytes produce proinflammatory cytokines (tumor necrosis factor α or TNFα, interleukin 10 or IL-10, interleukin-6 or IL-6, etc.), and nitric oxide (NO) that ultimately participate in autoimmune, inflammatory as well as neurodegenerative disorders like rheumatoid arthritis, multiple sclerosis, Alzheimer's disease, Parkinson's disease, Huntington's disease, traumatic brain injury, etc. Therefore, identification of nontoxic anti-inflammatory drugs may be beneficial for these autoimmune, inflammatory and neurodegenerative disorders. Cinnamein, an ester derivative of cinnamic acid and benzyl alcohol, is used as a flavoring agent and for its antifungal and antibacterial properties. Here, we demonstrate anti-inflammatory properties of cinnamein in RAW 264.7 macrophages and primary mouse microglia and astrocytes. Stimulation of RAW 264.7 macrophages with lipopolysaccharide (LPS) and interferon γ (IFNγ) led to marked production of NO (FIGS. 18A-C). However, cinnamein pretreatment for 6 h significantly inhibited LPS- and IFNγ-induced production of NO in RAW 264.7 macrophages (FIGS. 18A-C). Accordingly, LPS and viral double-stranded RNA mimic polyinosinic:polycytidylic acid (polyIC) stimulated the production of TNFα (FIGS. 19A-B), IL-10 (FIGS. 20A-B) and IL-6 (FIGS. 21A-B) in primary mouse microglia, which was strongly inhibited by cinnamein pretreatment. Similarly, cinnamein also inhibited polyIC-induced production of TNFα and IL-6 in primary mouse astrocytes (FIG. 22A-B). These results suggest that cinnamein may be used to control inflammation in different autoimmune, inflammatory and neurodegenerative disorders.

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other methods and systems for carrying out the several purposes of this disclosure. As a result, the claims should not be considered as limited by the description or examples.

1. A method of slowing the progression of or reducing the severity of a symptom associated with a nervous system injury in a subject in need thereof, the method comprising administering to the subject an effective amount of a benzoic acid salt or a prodrug thereof, thereby slowing the progression of or reducing the severity of the symptom associated with the nervous system injury.

2. The method of claim 1, wherein the benzoic acid salt, when present, is sodium benzoate, potassium benzoate, calcium benzoate, 2-aminobenzoate, 3-aminobenzoate, 4-aminobenzoate, or any combination thereof.

3. The method of claim 1, wherein the prodrug of the benzoic acid salt, when present, is benzyl cinnamate, glyceryl tribenzoate, cinnamic acid, benzyl acetate, benzyl alcohol, benzoic acid, quinic acid, phenylalanine, tyrosine, or any combination thereof.

4. The method of claim 1, wherein the subject has not been diagnosed with a urea cycle disorder, glycine encephalopathy, multiple sclerosis, Parkinson's disease, Alzheimer's disease, Huntington disease, or an autism spectrum disorder.

5. The method of claim 1, wherein the subject has been diagnosed with the nervous system injury prior to the administering step.

6. The method of claim 1, wherein the nervous system injury is a central nervous system (CNS) injury or a peripheral nerve injury.

7. The method of claim 1, wherein the nervous system injury is a spinal cord injury (SCI), spinal cord contusion, or nerve crush injury.

8. The method of claim 7, wherein the effective amount of the benzoic acid salt or the prodrug thereof is administered within 24 hours after the SCI, spinal cord contusion, or nerve crush injury.

9. The method of claim 1, wherein the nervous system injury is traumatic brain injury (TBI).

10. The method of claim 1, wherein the nervous system injury is demyelinating disorder.

11. The method of claim 10, wherein the demyelinating disorder is optic neuritis, X-Adrenoleukodystrophy, Krabbe disease, progressive multifocal leucoencephalopathy, adrenomyeloneuropathy, acute-disseminated encephalomyelitis, acute haemorrhagic leucoencephalitis, multiple sclerosis, Balo's disease (concentric sclerosis), Charcot-Marie-Tooth disease, Guillain-Barre syndrome, HTLV-I associated myelopathy, neuromyelitis optica (Devic's disease), Schilder's disease, transverse myelitis, or a combination thereof.

12. The method of claim 9, wherein the effective amount of the benzoic act salt or the prodrug thereof is administered within 24 hours after the traumatic brain injury.

13. The method of claim 1, wherein administering the effective amount of the benzoic acid salt or the prodrug thereof results in a reduction of glial inflammation, improvement in motor function or coordination, or an improvement in learning or memory dysfunction.

14. The method of claim 1, wherein administering the effective amount of the benzoic acid salt or the prodrug thereof prevents or reduces the severity of a symptom associated with mental depression.

15. The method of claim 1, wherein the benzoic acid salt or the prodrug thereof is administered as a pharmaceutical composition comprising the benzoic acid salt or the prodrug thereof and a pharmaceutically acceptable excipient; wherein the composition comprises greater than 0.10% of the benzoic acid salt or the prodrug thereof by weight of the composition.

16. The method of claim 1, wherein the benzoic acid salt or the prodrug thereof is administered orally.

17. A method of slowing the progression of or reducing the severity of a symptom associated with a nervous system injury in a subject in need thereof, the method comprising administering to the subject an effective amount of sodium benzoate, thereby slowing the progression of or reducing the severity of the symptom associated with the nervous system injury.

18. The method of claim 17, wherein the subject has not been diagnosed with a urea cycle disorder, glycine encephalopathy, multiple sclerosis, Parkinson's disease, Alzheimer's disease, Huntington disease, or an autism spectrum disorder.

19. The method of claim 17, wherein the subject has been diagnosed with the nervous system injury prior to the administering step.

20. The method of claim 17, wherein the nervous system injury is a central nervous system (CNS) injury or a peripheral nerve injury.

21-30. (canceled)
